(19) United States
(12) Patent Application Publication
(10) Pub. No.: US 2023/0210792 A1
(43) Pub. Date: Jul. 6, 2023

(54) USE OF A COMBINATION OF COLCHICINE AND A CXCR-2 INHIBITORS FOR THE TREATMENT OR PREVENTION OF FAMILIAL MEDITERRANEAN FEVER (FMF) AND FLARE-UPS THEREOF

(71) Applicant: ARISTEA THERAPEUTICS, INC., San Diego, CA (US)

(72) Inventors: Scott BAUMGARTNER, Spokane, WA (US); Nihar BHAKTA, Carlsbad, CA (US)

(21) Appl. No.: 18/000,504

(22) PCT Filed: Jun. 1, 2021
(86) PCT No.: PCT/US2021/035145

§ 371 Date: Dec. 1, 2022

Related U.S. Application Data
(60) Provisional application No. 63/035,500, filed on Jun. 5, 2020.

(51) Int. Cl. A61K 31/165; A61K 31/513; A61P 29/00

(52) U.S. Cl. CPC A61K 31/165; A61K 31/513; A61P 29/00

(57) ABSTRACT

The present invention relates to the treatment or prevention of Familial Mediterranean Fever (FMF) and flare-ups thereof comprising administering to the subject a combination of (i) colchicine, or a pharmaceutically acceptable salt thereof; and (ii) a CXCR-2 inhibitor, or a pharmaceutically acceptable salt thereof. The preferred CXCR-2 inhibitor N-(6-(((2R,3S)-3,4-dihydroxy butan-2-yl)oxy)-2-((4-fluorobenzyl)thio)pyrimidin-4-yl)-3-methylazetidine-1-sulfonamide.

### CROSS-REFERENCE

This application claims the benefit of U.S. Provisional Application No. 63/035,500, filed Jun. 5, 2020, which is incorporated herein by reference.

### BACKGROUND OF THE INVENTION

Chemokines play an important role in immune and inflammatory responses in various diseases and disorders. These small secreted molecules are a growing superfamily of 8-14 kDa proteins characterized by a conserved cysteine motif. The chemokine superfamily comprises three groups exhibiting characteristic structural motifs, the C—X—C, C—C and C—X₃—C families. The C—X—C chemokines include several potent chemo-attractants and activators of neutrophils.

### SUMMARY OF THE INVENTION

Disclosed herein is a method for treating Familial Mediterranean Fever (FMF) in a subject in need thereof, comprising administering to the subject a combination of: (i) Colchicine, or a pharmaceutically acceptable salt thereof; and (ii) a CXCR-2 inhibitor, or a pharmaceutically acceptable salt thereof. In some embodiments, the combination is a synergistic combination. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide:

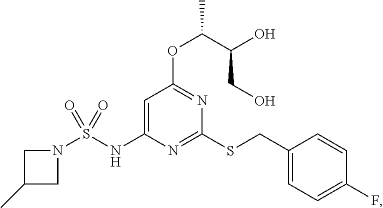 [SMILES: CC1CN(S(=O)(=O)Nc2cc(O[C@H](C)[C@@H](O)CO)nc(SCc3ccc(F)cc3)n2)C1]

or a pharmaceutically acceptable salt thereof. In some embodiments, the subject is colchicine-resistant. In some embodiments, colchicine-resistant means the subject has stopped responding to Colchicine treatment. In some embodiments, colchicine-resistant means more than 1 flare-up every 3 months despite treatment with 2 mg/day of colchicine. In some embodiments, colchicine-resistant means one flare-up or more per month in a subject who is receiving the maximally Colchicine tolerated dose for six months or more. In some embodiments, colchicine-resistant means more than 6 flare-ups per year. In some embodiments, colchicine-resistant means more than 3 flare-ups over 4-6 months. In some embodiments, the FMF flare-ups comprise fever, abdominal pain, joint pain, pleuritis, pericarditis, or any combinations thereof. In some embodiments, the FMF flare-ups comprise fever. In some embodiments, the FMF flare-ups comprise abdominal pain. In some embodiments, the FMF flare-ups comprise joint pain. In some embodiments, colchicine is administered in a therapeutically effective amount. In some embodiments, colchicine is administered in a sub-therapeutically effective amount. In some embodiments, the sub-therapeutic amount of colchicine results in fewer side effects than a therapeutic amount of colchicine. In some embodiments, the amount of Colchicine, or a pharmaceutically acceptable salt thereof, is between about 0.3 mg/day and about 3.0 mg/day. In some embodiments, the amount of Colchicine, or a pharmaceutically acceptable salt thereof, is between about 0.6 mg/day and about 2.4 mg/day. In some embodiments, the amount of Colchicine, or a pharmaceutically acceptable salt thereof, is between about 1.2 mg/day and about 2.4 mg/day. In some embodiments, the amount of Colchicine, or a pharmaceutically acceptable salt thereof, is about 0.6 mg/day. In some embodiments, the amount of Colchicine, or a pharmaceutically acceptable salt thereof, is about 1.2 mg/day. In some embodiments, the amount of Colchicine, or a pharmaceutically acceptable salt thereof, is about 2.4 mg/day. In some embodiments, the amount of Colchicine, or a pharmaceutically acceptable salt thereof, is about 0.5 mg/day. In some embodiments, the amount of Colchicine, or a pharmaceutically acceptable salt thereof, is about 1.0 mg/day. In some embodiments, the amount of Colchicine, or a pharmaceutically acceptable salt thereof, is about 1.5 mg/day. In some embodiments, the amount of Colchicine, or a pharmaceutically acceptable salt thereof, is about 2.0 mg/day. In some embodiments, the amount of Colchicine, or a pharmaceutically acceptable salt thereof, is about 2.5 mg/day. In some embodiments, the amount of Colchicine, or a pharmaceutically acceptable salt thereof, is about 3.0 mg/day. In some embodiments, the CXCR-2 inhibitor is administered in a therapeutically effective amount. In some embodiments, the CXCR-2 inhibitor is administered in a sub-therapeutically effective amount. In some embodiments, the amount of the CXCR-2 inhibitor, or a pharmaceutically acceptable salt thereof, is between about 10 mg/day and about 500 mg/day. In some embodiments, the amount of the CXCR-2 inhibitor, or a pharmaceutically acceptable salt thereof, is about 200 mg/day. In some embodiments, the amount of the CXCR-2 inhibitor, or a pharmaceutically acceptable salt thereof, is about 300 mg/day. In some embodiments, the amount of the CXCR-2 inhibitor, or a pharmaceutically acceptable salt thereof, is about 400 mg/day. In some embodiments, the CXCR-2 inhibitor, or a pharmaceutically acceptable salt thereof, and Colchicine, or a pharmaceutically acceptable salt thereof are formulated in the same pharmaceutical composition. In some embodiments, the pharmaceutical composition is a solution, suspension, tablet, or capsule. In some embodiments, the pharmaceutical composition is formulated as a sustained release or delayed release formulation. In some embodiments, the CXCR-2 inhibitor, or a pharmaceutically acceptable salt thereof, and Colchicine, or a pharmaceutically acceptable salt thereof are formulated in different pharmaceutical compositions. In some embodiments, the Colchicine pharmaceutical composition is a solution, suspension, tablet, or capsule. In some embodiments, the Colchicine pharmaceutical composition is formulated as a sustained release or delayed release formulation. In some embodiments, the CXCR-2 inhibitor pharmaceutical composition is a solution, suspension, tablet, or capsule. In some embodiments, the CXCR-2 inhibitor pharmaceutical composition is formulated as a sustained release or delayed release formulation. In some embodiments, the CXCR-2 inhibitor and Colchicine are administered concomitantly. In some embodiments, the CXCR-2 inhibitor and Colchicine are administered sequentially.

In some embodiments, the FMF flare-up is an acute FMF flare-up. In some embodiments, the subject presents with a C-reactive protein (CRP) level of greater than 10 mg/L.

Disclosed herein is a method for preventing Familial Mediterranean Fever (FMF) flare-ups in a subject having been diagnosed with FMF, comprising administering to the subject a combination of: (i) Colchicine, or a pharmaceutically acceptable salt thereof; and (ii) a CXCR-2 inhibitor, or a pharmaceutically acceptable salt thereof. In some embodiments, the combination is a synergistic combination. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide:

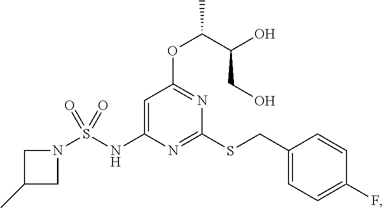 [SMILES: CC1CN(S(=O)(=O)Nc2cc(O[C@H](C)[C@@H](O)CO)nc(SCc3ccc(F)cc3)n2)C1]

or a pharmaceutically acceptable salt thereof. In some embodiments, the subject is colchicine-resistant. In some embodiments, colchicine-resistant means the subject has stopped responding to Colchicine treatment. In some embodiments, colchicine-resistant means more than 1 flare-up every 3 months despite treatment with 2 mg/day of colchicine. In some embodiments, colchicine-resistant means one flare-up or more per month in a subject who is receiving the maximally Colchicine tolerated dose for six months or more. In some embodiments, colchicine-resistant means more than 6 flare-ups per year. In some embodiments, colchicine-resistant means more than 3 flare-ups over 4-6 months. In some embodiments, the FMF flare-ups comprise fever, abdominal pain, joint pain, pleuritis, pericarditis, or any combinations thereof. In some embodiments, the FMF flare-ups comprise fever. In some embodiments, the FMF flare-ups comprise abdominal pain. In some embodiments, the FMF flare-ups comprise joint pain. In some embodiments, colchicine is administered in a therapeutically effective amount. In some embodiments, colchicine is administered in a sub-therapeutically effective amount. In some embodiments, the sub-therapeutic amount of colchicine results in fewer side effects than a therapeutic amount of colchicine. In some embodiments, the amount of Colchicine, or a pharmaceutically acceptable salt thereof, is between about 0.3 mg/day and about 3.0 mg/day. In some embodiments, the amount of Colchicine, or a pharmaceutically acceptable salt thereof, is between about 0.6 mg/day and about 2.4 mg/day. In some embodiments, the amount of Colchicine, or a pharmaceutically acceptable salt thereof, is between about 1.2 mg/day and about 2.4 mg/day. In some embodiments, the amount of Colchicine, or a pharmaceutically acceptable salt thereof, is about 0.6 mg/day. In some embodiments, the amount of Colchicine, or a pharmaceutically acceptable salt thereof, is about 1.2 mg/day. In some embodiments, the amount of Colchicine, or a pharmaceutically acceptable salt thereof, is about 2.4 mg/day. In some embodiments, the amount of Colchicine, or a pharmaceutically acceptable salt thereof, is about 0.5 mg/day. In some embodiments, the amount of Colchicine, or a pharmaceutically acceptable salt thereof, is about 1.0 mg/day. In some embodiments, the amount of Colchicine, or a pharmaceutically acceptable salt thereof, is about 1.5 mg/day. In some embodiments, the amount of Colchicine, or a pharmaceutically acceptable salt thereof, is about 2.0 mg/day. In some embodiments, the amount of Colchicine, or a pharmaceutically acceptable salt thereof, is about 2.5 mg/day. In some embodiments, the amount of Colchicine, or a pharmaceutically acceptable salt thereof, is about 3.0 mg/day. In some embodiments, the CXCR-2 inhibitor is administered in a therapeutically effective amount. In some embodiments, the CXCR-2 inhibitor is administered in a sub-therapeutically effective amount. In some embodiments, the amount of the CXCR-2 inhibitor, or a pharmaceutically acceptable salt thereof, is between about 10 mg/day and about 500 mg/day. In some embodiments, the amount of the CXCR-2 inhibitor, or a pharmaceutically acceptable salt thereof, is about 200 mg/day. In some embodiments, the amount of the CXCR-2 inhibitor, or a pharmaceutically acceptable salt thereof, is about 300 mg/day. In some embodiments, the amount of the CXCR-2 inhibitor, or a pharmaceutically acceptable salt thereof, is about 400 mg/day. In some embodiments, the CXCR-2 inhibitor, or a pharmaceutically acceptable salt thereof, and Colchicine, or a pharmaceutically acceptable salt thereof are formulated in the same pharmaceutical composition. In some embodiments, the pharmaceutical composition is a solution, suspension, tablet, or capsule. In some embodiments, the pharmaceutical composition is formulated as a sustained release or delayed release formulation. In some embodiments, the CXCR-2 inhibitor, or a pharmaceutically acceptable salt thereof, and Colchicine, or a pharmaceutically acceptable salt thereof are formulated in different pharmaceutical compositions. In some embodiments, the Colchicine pharmaceutical composition is a solution, suspension, tablet, or capsule. In some embodiments, the Colchicine pharmaceutical composition is formulated as a sustained release or delayed release formulation. In some embodiments, the CXCR-2 inhibitor pharmaceutical composition is a solution, suspension, tablet, or capsule. In some embodiments, the CXCR-2 inhibitor pharmaceutical composition is formulated as a sustained release or delayed release formulation. In some embodiments, the CXCR-2 inhibitor and Colchicine are administered concomitantly. In some embodiments, the CXCR-2 inhibitor and Colchicine are administered sequentially.

In some embodiments, the FMF flare-up is an acute FMF flare-up. In some embodiments, the subject presents with a C-reactive protein (CRP) level of greater than 10 mg/L.

Disclosed herein is a method for treating acute Familial Mediterranean Fever (FMF) flare-ups in a subject having been diagnosed with FMF, comprising administering to the subject a combination of: (i) Colchicine, or a pharmaceutically acceptable salt thereof; and (ii) a CXCR-2 inhibitor, or a pharmaceutically acceptable salt thereof. In some embodiments, the combination is a synergistic combination. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide:

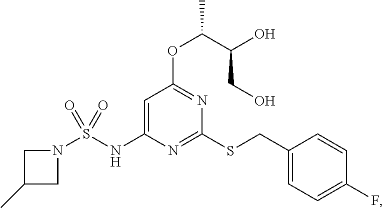 [SMILES: CC1CN(S(=O)(=O)Nc2cc(O[C@H](C)[C@@H](O)CO)nc(SCc3ccc(F)cc3)n2)C1]

or a pharmaceutically acceptable salt thereof. In some embodiments, the subject is colchicine-resistant. In some embodiments, colchicine-resistant means the subject has stopped responding to Colchicine treatment. In some embodiments, colchicine-resistant means more than 1 flare-up every 3 months despite treatment with 2 mg/day of colchicine. In some embodiments, colchicine-resistant means one flare-up or more per month in a subject who is receiving the maximally Colchicine tolerated dose for six months or more. In some embodiments, colchicine-resistant means more than 6 flare-ups per year. In some embodiments, colchicine-resistant means more than 3 flare-ups over 4-6 months. In some embodiments, the FMF flare-ups comprise fever, abdominal pain, joint pain, pleuritis, pericarditis, or any combinations thereof. In some embodiments, the FMF flare-ups comprise fever. In some embodiments, the FMF flare-ups comprise abdominal pain. In some embodiments, the FMF flare-ups comprise joint pain. In some embodiments, colchicine is administered in a therapeutically effective amount. In some embodiments, colchicine is administered in a sub-therapeutically effective amount. In some embodiments, the sub-therapeutic amount of colchicine results in fewer side effects than a therapeutic amount of colchicine. In some embodiments, the amount of Colchicine, or a pharmaceutically acceptable salt thereof, is between about 0.3 mg/day and about 3.0 mg/day. In some embodiments, the amount of Colchicine, or a pharmaceutically acceptable salt thereof, is between about 0.6 mg/day and about 2.4 mg/day. In some embodiments, the amount of Colchicine, or a pharmaceutically acceptable salt thereof, is between about 1.2 mg/day and about 2.4 mg/day. In some embodiments, the amount of Colchicine, or a pharmaceutically acceptable salt thereof, is about 0.6 mg/day. In some embodiments, the amount of Colchicine, or a pharmaceutically acceptable salt thereof, is about 1.2 mg/day. In some embodiments, the amount of Colchicine, or a pharmaceutically acceptable salt thereof, is about 2.4 mg/day. In some embodiments, the amount of Colchicine, or a pharmaceutically acceptable salt thereof, is about 0.5 mg/day. In some embodiments, the amount of Colchicine, or a pharmaceutically acceptable salt thereof, is about 1.0 mg/day. In some embodiments, the amount of Colchicine, or a pharmaceutically acceptable salt thereof, is about 1.5 mg/day. In some embodiments, the amount of Colchicine, or a pharmaceutically acceptable salt thereof, is about 2.0 mg/day. In some embodiments, the amount of Colchicine, or a pharmaceutically acceptable salt thereof, is about 2.5 mg/day. In some embodiments, the amount of Colchicine, or a pharmaceutically acceptable salt thereof, is about 3.0 mg/day. In some embodiments, the CXCR-2 inhibitor is administered in a therapeutically effective amount. In some embodiments, the CXCR-2 inhibitor is administered in a sub-therapeutically effective amount. In some embodiments, the amount of the CXCR-2 inhibitor, or a pharmaceutically acceptable salt thereof, is between about 10 mg/day and about 500 mg/day. In some embodiments, the amount of the CXCR-2 inhibitor, or a pharmaceutically acceptable salt thereof, is about 200 mg/day. In some embodiments, the amount of the CXCR-2 inhibitor, or a pharmaceutically acceptable salt thereof, is about 300 mg/day. In some embodiments, the amount of the CXCR-2 inhibitor, or a pharmaceutically acceptable salt thereof, is about 400 mg/day. In some embodiments, the CXCR-2 inhibitor, or a pharmaceutically acceptable salt thereof, and Colchicine, or a pharmaceutically acceptable salt thereof are formulated in the same pharmaceutical composition. In some embodiments, the pharmaceutical composition is a solution, suspension, tablet, or capsule. In some embodiments, the pharmaceutical composition is formulated as a sustained release or delayed release formulation. In some embodiments, the CXCR-2 inhibitor, or a pharmaceutically acceptable salt thereof, and Colchicine, or a pharmaceutically acceptable salt thereof are formulated in different pharmaceutical compositions. In some embodiments, the Colchicine pharmaceutical composition is a solution, suspension, tablet, or capsule. In some embodiments, the Colchicine pharmaceutical composition is formulated as a sustained release or delayed release formulation. In some embodiments, the CXCR-2 inhibitor pharmaceutical composition is a solution, suspension, tablet, or capsule. In some embodiments, the CXCR-2 inhibitor pharmaceutical composition is formulated as a sustained release or delayed release formulation. In some embodiments, the CXCR-2 inhibitor and Colchicine are administered concomitantly. In some embodiments, the CXCR-2 inhibitor and Colchicine are administered sequentially.

In some embodiments, the FMF flare-up is an acute FMF flare-up. In some embodiments, the subject presents with a C-reactive protein (CRP) level of greater than 10 mg/L.

Disclosed herein is a method for decreasing the severity of Familial Mediterranean Fever (FMF) flare-ups in a subject having been diagnosed with FMF, comprising administering to the subject a combination of: (i) Colchicine, or a pharmaceutically acceptable salt thereof, and (ii) a CXCR-2 inhibitor, or a pharmaceutically acceptable salt thereof. In some embodiments, of a method of decreasing the severity of Familial Mediterranean Fever (FMF) flare-ups, the severity of the FMF flare-ups is decreased as compared to a subject being administered Colchicine alone. In some embodiments, of a method of decreasing the severity of Familial Mediterranean Fever (FMF) flare-ups, the severity of the FMF flare-ups is decreased as compared to a subject receiving no therapy. In some embodiments, of a method of decreasing the severity of Familial Mediterranean Fever (FMF) flare-ups, the severity of the flare-ups is decreased by at least about 50%. In some embodiments, of a method of decreasing the severity of Familial Mediterranean Fever (FMF) flare-ups, the severity of the flare-ups is decreased by at least about 70%. In some embodiments, of a method of decreasing the severity of Familial Mediterranean Fever (FMF) flare-ups, the severity of the flare-ups is decreased by at least about 90%. In some embodiments, of a method of decreasing the severity of Familial Mediterranean Fever (FMF) flare-ups, the severity of the flare-ups is decreased by at least about 99%. In some embodiments, the combination is a synergistic combination. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methyl azetidine-1l-sulfonamide:

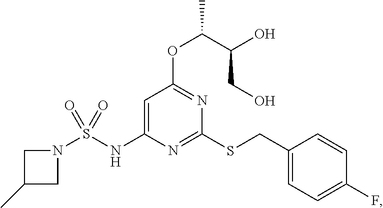 [SMILES: CC1CN(S(=O)(=O)Nc2cc(O[C@H](C)[C@@H](O)CO)nc(SCc3ccc(F)cc3)n2)C1]

or a pharmaceutically acceptable salt thereof. In some embodiments, the subject is colchicine-resistant. In some embodiments, colchicine-resistant means the subject has stopped responding to Colchicine treatment. In some embodiments, colchicine-resistant means more than 1 flare-up every 3 months despite treatment with 2 mg/day of colchicine. In some embodiments, colchicine-resistant means one flare-up or more per month in a subject who is receiving the maximally Colchicine tolerated dose for six months or more. In some embodiments, colchicine-resistant means more than 6 flare-ups per year. In some embodiments, colchicine-resistant means more than 3 flare-ups over 4-6 months. In some embodiments, the FMF flare-ups comprise fever, abdominal pain, joint pain, pleuritis, pericarditis, or any combinations thereof. In some embodiments, the FMF flare-ups comprise fever. In some embodiments, the FMF flare-ups comprise abdominal pain. In some embodiments, the FMF flare-ups comprise joint pain. In some embodiments, colchicine is administered in a therapeutically effective amount. In some embodiments, colchicine is administered in a sub-therapeutically effective amount. In some embodiments, the sub-therapeutic amount of colchicine results in fewer side effects than a therapeutic amount of colchicine. In some embodiments, the amount of Colchicine, or a pharmaceutically acceptable salt thereof, is between about 0.3 mg/day and about 3.0 mg/day. In some embodiments, the amount of Colchicine, or a pharmaceutically acceptable salt thereof, is between about 0.6 mg/day and about 2.4 mg/day. In some embodiments, the amount of Colchicine, or a pharmaceutically acceptable salt thereof, is between about 1.2 mg/day and about 2.4 mg/day. In some embodiments, the amount of Colchicine, or a pharmaceutically acceptable salt thereof, is about 0.6 mg/day. In some embodiments, the amount of Colchicine, or a pharmaceutically acceptable salt thereof, is about 1.2 mg/day. In some embodiments, the amount of Colchicine, or a pharmaceutically acceptable salt thereof, is about 2.4 mg/day. In some embodiments, the amount of Colchicine, or a pharmaceutically acceptable salt thereof, is about 0.5 mg/day. In some embodiments, the amount of Colchicine, or a pharmaceutically acceptable salt thereof, is about 1.0 mg/day. In some embodiments, the amount of Colchicine, or a pharmaceutically acceptable salt thereof, is about 1.5 mg/day. In some embodiments, the amount of Colchicine, or a pharmaceutically acceptable salt thereof, is about 2.0 mg/day. In some embodiments, the amount of Colchicine, or a pharmaceutically acceptable salt thereof, is about 2.5 mg/day. In some embodiments, the amount of Colchicine, or a pharmaceutically acceptable salt thereof, is about 3.0 mg/day. In some embodiments, the CXCR-2 inhibitor is administered in a therapeutically effective amount. In some embodiments, the CXCR-2 inhibitor is administered in a sub-therapeutically effective amount. In some embodiments, the amount of the CXCR-2 inhibitor, or a pharmaceutically acceptable salt thereof, is between about 10 mg/day and about 500 mg/day. In some embodiments, the amount of the CXCR-2 inhibitor, or a pharmaceutically acceptable salt thereof, is about 200 mg/day. In some embodiments, the amount of the CXCR-2 inhibitor, or a pharmaceutically acceptable salt thereof, is about 300 mg/day. In some embodiments, the amount of the CXCR-2 inhibitor, or a pharmaceutically acceptable salt thereof, is about 400 mg/day. In some embodiments, the CXCR-2 inhibitor, or a pharmaceutically acceptable salt thereof, and Colchicine, or a pharmaceutically acceptable salt thereof are formulated in the same pharmaceutical composition. In some embodiments, the pharmaceutical composition is a solution, suspension, tablet, or capsule. In some embodiments, the pharmaceutical composition is formulated as a sustained release or delayed release formulation. In some embodiments, the CXCR-2 inhibitor, or a pharmaceutically acceptable salt thereof, and Colchicine, or a pharmaceutically acceptable salt thereof are formulated in different pharmaceutical compositions. In some embodiments, the Colchicine pharmaceutical composition is a solution, suspension, tablet, or capsule. In some embodiments, the Colchicine pharmaceutical composition is formulated as a sustained release or delayed release formulation. In some embodiments, the CXCR-2 inhibitor pharmaceutical composition is a solution, suspension, tablet, or capsule. In some embodiments, the CXCR-2 inhibitor pharmaceutical composition is formulated as a sustained release or delayed release formulation. In some embodiments, the CXCR-2 inhibitor and Colchicine are administered concomitantly. In some embodiments, the CXCR-2 inhibitor and Colchicine are administered sequentially.

In some embodiments, the FMF flare-up is an acute FMF flare-up. In some embodiments, the subject presents with a C-reactive protein (CRP) level of greater than 10 mg/L.

Disclosed herein is a method for decreasing the occurrence of Familial Mediterranean Fever (FMF) flare-ups in a subject having been diagnosed with FMF, comprising administering to the subject a combination of: (i) Colchicine, or a pharmaceutically acceptable salt thereof; and (ii) a CXCR-2 inhibitor, or a pharmaceutically acceptable salt thereof. In some embodiments, of a method of decreasing the occurrence of Familial Mediterranean Fever (FMF) flare-ups, the occurrence of the FMF flare-ups is decreased as compared to a subject being administered Colchicine alone. In some embodiments, of a method of decreasing the occurrence of Familial Mediterranean Fever (FMF) flare-ups, the occurrence of the FMF flare-ups is decreased as compared to a subject receiving no therapy. In some embodiments, of a method of decreasing the occurrence of Familial Mediterranean Fever (FMF) flare-ups, the occurrence of the flare-ups is decreased by at least about 50%. In some embodiments, of a method of decreasing the occurrence of Familial Mediterranean Fever (FMF) flare-ups, the occurrence of the flare-ups is decreased by at least about 70%. In some embodiments, of a method of decreasing the occurrence of Familial Mediterranean Fever (FMF) flare-ups, the occurrence of the flare-ups is decreased by at least about 90%. In some embodiments, of a method of decreasing the occurrence of Familial Mediterranean Fever (FMF) flare-ups, the occurrence of the flare-ups is decreased by at least about 99%. In some embodiments, the combination is a synergistic combination. In some embodiments, the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide:

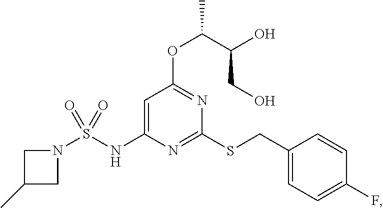 [SMILES: CC1CN(S(=O)(=O)Nc2cc(O[C@H](C)[C@@H](O)CO)nc(SCc3ccc(F)cc3)n2)C1]

or a pharmaceutically acceptable salt thereof. In some embodiments, the subject is colchicine-resistant. In some embodiments, colchicine-resistant means the subject has stopped responding to Colchicine treatment. In some embodiments, colchicine-resistant means more than 1 flare-up every 3 months despite treatment with 2 mg/day of colchicine. In some embodiments, colchicine-resistant means one flare-up or more per month in a subject who is receiving the maximally Colchicine tolerated dose for six months or more. In some embodiments, colchicine-resistant means more than 6 flare-ups per year. In some embodiments, colchicine-resistant means more than 3 flare-ups over 4-6 months. In some embodiments, the FMF flare-ups comprise fever, abdominal pain, joint pain, pleuritis, pericarditis, or any combinations thereof. In some embodiments, the FMF flare-ups comprise fever. In some embodiments, the FMF flare-ups comprise abdominal pain. In some embodiments, the FMF flare-ups comprise joint pain. In some embodiments, colchicine is administered in a therapeutically effective amount. In some embodiments, colchicine is administered in a sub-therapeutically effective amount. In some embodiments, the sub-therapeutic amount of colchicine results in fewer side effects than a therapeutic amount of colchicine. In some embodiments, the amount of Colchicine, or a pharmaceutically acceptable salt thereof, is between about 0.3 mg/day and about 3.0 mg/day. In some embodiments, the amount of Colchicine, or a pharmaceutically acceptable salt thereof, is between about 0.6 mg/day and about 2.4 mg/day. In some embodiments, the amount of Colchicine, or a pharmaceutically acceptable salt thereof, is between about 1.2 mg/day and about 2.4 mg/day. In some embodiments, the amount of Colchicine, or a pharmaceutically acceptable salt thereof, is about 0.6 mg/day. In some embodiments, the amount of Colchicine, or a pharmaceutically acceptable salt thereof, is about 1.2 mg/day. In some embodiments, the amount of Colchicine, or a pharmaceutically acceptable salt thereof, is about 2.4 mg/day. In some embodiments, the amount of Colchicine, or a pharmaceutically acceptable salt thereof, is about 0.5 mg/day. In some embodiments, the amount of Colchicine, or a pharmaceutically acceptable salt thereof, is about 1.0 mg/day. In some embodiments, the amount of Colchicine, or a pharmaceutically acceptable salt thereof, is about 1.5 mg/day. In some embodiments, the amount of Colchicine, or a pharmaceutically acceptable salt thereof, is about 2.0 mg/day. In some embodiments, the amount of Colchicine, or a pharmaceutically acceptable salt thereof, is about 2.5 mg/day. In some embodiments, the amount of Colchicine, or a pharmaceutically acceptable salt thereof, is about 3.0 mg/day. In some embodiments, the CXCR-2 inhibitor is administered in a therapeutically effective amount. In some embodiments, the CXCR-2 inhibitor is administered in a sub-therapeutically effective amount. In some embodiments, the amount of the CXCR-2 inhibitor, or a pharmaceutically acceptable salt thereof, is between about 10 mg/day and about 500 mg/day. In some embodiments, the amount of the CXCR-2 inhibitor, or a pharmaceutically acceptable salt thereof, is about 200 mg/day. In some embodiments, the amount of the CXCR-2 inhibitor, or a pharmaceutically acceptable salt thereof, is about 300 mg/day. In some embodiments, the amount of the CXCR-2 inhibitor, or a pharmaceutically acceptable salt thereof, is about 400 mg/day. In some embodiments, the CXCR-2 inhibitor, or a pharmaceutically acceptable salt thereof, and Colchicine, or a pharmaceutically acceptable salt thereof are formulated in the same pharmaceutical composition. In some embodiments, the pharmaceutical composition is a solution, suspension, tablet, or capsule. In some embodiments, the pharmaceutical composition is formulated as a sustained release or delayed release formulation. In some embodiments, the CXCR-2 inhibitor, or a pharmaceutically acceptable salt thereof, and Colchicine, or a pharmaceutically acceptable salt thereof are formulated in different pharmaceutical compositions. In some embodiments, the Colchicine pharmaceutical composition is a solution, suspension, tablet, or capsule. In some embodiments, the Colchicine pharmaceutical composition is formulated as a sustained release or delayed release formulation. In some embodiments, the CXCR-2 inhibitor pharmaceutical composition is a solution, suspension, tablet, or capsule. In some embodiments, the CXCR-2 inhibitor pharmaceutical composition is formulated as a sustained release or delayed release formulation. In some embodiments, the CXCR-2 inhibitor and Colchicine are administered concomitantly. In some embodiments, the CXCR-2 inhibitor and Colchicine are administered sequentially.

In some embodiments, the FMF flare-up is an acute FMF flare-up. In some embodiments, the subject presents with a C-reactive protein (CRP) level of greater than 10 mg/L.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the study design of the phase II study.

FIG. 2 shows the steps for addressing neutrophil counts during the phase II study.

### DETAILED DESCRIPTION OF THE INVENTION

### Definitions

The term “subject”, as used herein in reference to individuals suffering from a disorder, and the like, encompasses mammals and non-mammals. In one embodiment of the methods and compositions provided herein, the mammal is a human.

The terms “effective amount”, “therapeutically effective amount” or “pharmaceutically effective amount” as used herein, refer to an amount of at least one agent or compound being administered that is sufficient to treat or prevent the particular disease or condition. The result is the reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case is determined using techniques such as a dose escalation study.

A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

A “synergistically effective” therapeutic amount of an agent or therapy is an amount that, when combined with an effective or sub-therapeutic amount of another agent or therapy, produces a greater effect than the expected additive effects of each of the two agents or therapies. The term “greater effect” encompasses not only a reduction in symptoms of the disorder to be treated, but also an improved side effect profile, improved tolerability, improved patient compliance, improved efficacy, or any other improved clinical outcome.

The terms “synergistic” and “synergistically” as applied to the effect of two or more pharmaceutically active ingredients used in combination (whether simultaneously or sequentially) refer to a greater effect than the expected additive effects of the two agents.

The term “about” refers to ±10% of a stated number or value.

In the context of the present specification, the term “therapy” also includes “prophylaxis” unless there are specific indications to the contrary. The terms “therapeutic” and “therapeutically” should be construed accordingly.

The term “Colchicine Resistant” or “Colchicine Resistance” is defined as can be seen in the table below:

| Reference | Main criteria |
|---|---|
| Semin Arthritis | Non-responders defined as patients with >1 clinical attack every 3 |
| Rheum, 33 (2004), | months despite treatment with 2 mg/day of colchicine |
| pp. 273-282 | |
| Clin Exp Rheumatol, | FMF response defined based on the % reduction in attack frequency; |
| 26 (4 Suppl. 50) | patients not reaching an FMF-50 response would be classified as non- |
| (2008), pp. S49-S51 | responders |
| Semin Arthritis | Fully compliant patients considered colchicine resistant if they have >6 |
| Rheum, 43 (2013), | typical FMF attacks per year or >3 attacks over 4-6 months |
| pp. 387-391 | |
| Ann Rheum Dis, 73 | Beyond attack frequency, FMF-50 now also requires ≥50% |
| (2014), pp. 897-901 | improvement in 5 of 6 criteria; patients not achieving FMF-50 response |
| | considered to be colchicine resistant. 6 criteria include: % change in |
| | frequency of attacks, % change in duration of attacks, patient/parent |
| | global assessment of disease severity, physician global assessment of |
| | disease severity, % change in arthritis attacks, and % change in acute |
| | phase reactants |
| Ann Rheum Dis, 75 | Colchicine resistance defined by ≥1 attacks per month in compliant |
| (2016), pp. 644-651 | patients receiving the maximally tolerated dose for ≥6 months |

In some embodiments, colchicine-resistant means the subject has stopped responding to Colchicine treatment. In some embodiments, colchicine-resistant means more than 1 flare-up every 3 months despite treatment with 2 mg/day of colchicine. In some embodiments, colchicine-resistant means one flare-up or more per month in a subject who is receiving the maximally Colchicine tolerated dose for six months or more. In some embodiments, colchicine-resistant means more than 6 flare-ups per year. In some embodiments, colchicine-resistant means more than 3 flare-ups over 4-6 months.

### Agents

### Colchicine

Colchicine is the first line labelled and approved therapy for treating Familial Mediterranean Fever (FMF). Colchicine is primarily effective as a prophylactic treatment for the FMF attacks. It is recommended in all patients regardless of the frequency and intensity of attacks. Use of intermittent high-dose colchicine for treatment of acute attacks of FMF only is not recommended since it does not protect from the development of amyloidosis resulting from low-grade inflammation that can occur during asymptomatic intervals.

Since 1972 colchicine has become the drug of choice for prophylaxis against FMF attacks and FMF-associated amyloidosis. Colchicine, an alkaloid neutral, is absorbed in the jejunum and ileum. Colchicine is able to prevent activation of neutrophils, binding beta-tubulin and making beta-tubulin-colchicine complexes; inhibiting assembly of microtubules and mitotic spindle formation. Colchicine's mode of action includes modulation of chemokines, prostanoids production, inhibition of neutrophil and endothelial cell adhesion molecules.

Colchicine is rapidly absorbed from the gastrointestinal tract. Peak concentrations occur in 0.5 to 2 hours. The drug and its metabolites are distributed in leukocytes, kidneys, liver, spleen and the intestinal tract. Colchicine is metabolized in the liver and excreted primarily in the feces with 10-20% eliminated unchanged in the urine.

Common side effects from taking Colchicine include diarrhea, nausea, vomiting, abdominal pain and pharyngolaryngeal pain. Warnings regarding the use of Colchicine include blood dyscrasias (myelosuppression, leukopenia, granulocytopenia, thrombocytopenia and aplastic anemia); drug interaction with P-gp and/or CYP3A4 inhibitors (resulting in life-threatening interactions and death) and neuromuscular toxicity (myotoxicity including rhabdomyolysis).

The most frequently reported adverse side effects to Colchicine therapy are gastrointestinal, specifically diarrhea; abdominal pain with cramps; nausea; and vomiting. Less frequently or rarely reported adverse side effects associated with colchicine therapy include anorexia, agranulocytosis, allergic dermatitis, allergic reactions, alopecia, angioedema, aplastic anemia, bone marrow depression, myopathy, neuropathy, skin rash, thrombocytopenic disorder and urticaria.

### Colchicine Dosing

The amount of colchicine administered will firstly be dependent on the mammal being treated. In some embodiments, the daily dosage will normally be determined by the prescribing physician with the dosage generally varying according to the age, sex, diet, weight, general health and response of the individual patient, the severity of the patient's symptoms, the precise indication or condition being treated, the severity of the indication or condition being treated, time of administration, route of administration, drug absorption, the disposition of the composition, rate of excretion, drug combination, and the discretion of the prescribing physician. Also, the route of administration varies depending on the condition and its severity. The pharmaceutical composition is, in some embodiments, in unit dosage form. In such form, the preparation is subdivided into unit doses containing appropriate quantities of the active component, e.g., an effective amount to achieve the desired purpose. Determination of the proper dosage for a particular situation is within the skill of the art. In some embodiments, the total daily dosage is divided and administered in portions during the day if desired. The amount and frequency of administration will be regulated according to the judgment of the attending clinician physician considering such factors as described above. Thus, the amount of pharmaceutical composition to be administered is variable depending upon the circumstances. In some instances, dosage levels below the lower limit of the aforesaid range are more than adequate, while in other cases still larger doses are employed without causing any harmful side effect, e.g. by dividing such larger doses into several small doses for administration throughout the day. In combinational applications in which the compound is not the sole therapy, it is possible to administer lesser amounts of compound and still have therapeutic or prophylactic effect.

In some embodiments, the methods comprise administering between about 0.3 mg/day and about 3.0 mg/day of colchicine. In some embodiments, the methods comprise administering between about 1.2 mg/day and about 2.4 mg/day of colchicine. In some embodiments, the methods comprise administering between about 0.3 mg/day and about 3.0 mg/day of colchicine.

In some embodiments, the methods comprise administering about 3.0 mg, about 2.9 mg, about 2.8 mg, about 2.7 mg, about 2.6 mg, about 2.5 mg, about 2.4 mg, about 2.3 mg, about 2.2 mg, about 2.1 mg, about 2.0 mg, about 1.9 mg, about 1.8 mg, about 1.7 mg, about 1.6 mg, about 1.5 mg, about 1.4 mg, about 1.3 mg, about 1.2 mg, about 1.1 mg, about 1.0 mg, about 0.9 mg, about 0.8 mg, about 0.7 mg, about 0.6 mg, about 0.5 mg, about 0.4 mg, or about 0.3 mg per day of colchicine. In some embodiments, the methods comprise administering about 2.4 mg per day of colchicine. In some embodiments, the methods comprise administering about 1.2 mg per day of colchicine. In some embodiments, the methods comprise administering about 0.6 mg per day of colchicine. In some embodiments, the methods comprise administering about 0.5 mg per day of colchicine. In some embodiments, the methods comprise administering about 1.0 mg per day of colchicine. In some embodiments, the methods comprise administering about 1.5 mg per day of colchicine. In some embodiments, the methods comprise administering about 2.0 mg per day of colchicine. In some embodiments, the methods comprise administering about 2.5 mg per day of colchicine. In some embodiments, the methods comprise administering about 3.0 mg per day of colchicine.

### CXCR-2 Inhibitors

Chemokines play an important role in immune and inflammatory responses in various diseases and disorders. These small secreted molecules are a growing superfamily of 8-14 kDa proteins characterized by a conserved cysteine motif.

The chemokine superfamily comprises three groups exhibiting characteristic structural motifs, the C—X—C, C—C and C—X₃—C families. The C—X—C chemokines include several potent chemo-attractants and activators of neutrophils such as interleukin-8 (IL-8) and neutrophil-activating peptide 2 (NAP-2).

Studies have demonstrated that the actions of the chemokines are mediated by subfamilies of G protein-coupled receptors, among which are the receptors designated CXCR-1, CXCR-2, CXCR-3, CXCR-4 and CXCR-5 (for the C—X—C family). Only IL-8, and certain other C—X—C chemokines that bind IL-8 receptors, are known to chemo-attract human neutrophils. Only CXCR2 ligands such as IL-8 and CXCL-1(gro-alpa) are known to chemo-attract human neutrophils. C—X—C chemokines that chemo-attract neutrophils share specific sequence motifs. These receptors represent good targets for drug development since agents that modulate these receptors would be useful in the treatment of immune and inflammatory related disorders and diseases, such as Familial Mediterraneen Fever (FMF).

CXCR-2 is an IL-8 receptor. Chemokines that bind CXCR-2 are required for neutrophilic inflammation, for example in acute gout (Terkaltaub et al, Arthritis & Rheumatism, (1988), Vol 41, (No 5) pp 900-909). Urate crystals can initiate, amplify and sustain an intense inflammatory attack because they stimulate the synthesis and release of humoral and cellular inflammatory mediators. Neutrophilic synovitis is the hallmark of an acute gouty attack. Neutrophils are rare in normal synovial fluid. Monosodium urate monohydrate (MSUM) crystals from supersaturated extracellular fluids are deposited in synovial tissue, which activates resident mononuclear phagocytes and synovial lining cells to release neutrophil chemotaxins—CXCR-2 ligand, including IL-8. The newly generated neutrophil chemotaxins direct neutrophil transmigration. MSUM crystals interact with the phagocyte through two broad mechanisms. First, the crystals activate cells as opsonized and phagocytosed particles, eliciting the phagocyte response and release of inflammatory mediators. Second, urate crystals interact directly with lipid membranes and proteins, leading to the activation of several signal transduction pathways. These steps are critical for crystal-induced IL-8 expression. IL-8 is abundant in the synovial fluid in both acute gout and pseudogout. The rapid release of IL-8 (and other neutrophil chemotactic C—X—C chemokines) by crystal-activated resident mononuclear phagocytes and synovial lining cells triggers acute gout. Once in the synovial tissue, the neutrophils follow concentration gradients of chemoattractants such as C5a, leukotriene B4, platelet-activating factor, IL-1, and IL-8. Of these factors, IL-8 plays a central role in neutrophil invasion, accounting for approximately 90% of the neutrophil chemotactic activity of monocytes in response to urate crystals.

In some embodiments, neutralization of IL-8 or its receptor CXCR-2 substantially reduces the IL-8-induced neutrophilic inflammatory process and provide a potential therapeutic target for FMF.

In some embodiments, the following compounds provided in the following table, or pharmaceutically acceptable salts thereof, may be useful to treat diseases in which the chemokine receptor belongs to the CXC chemokine receptor subfamily, more conveniently the target chemokine receptor is the CXCR-2 receptor. In some embodiments, the compounds 1, 2, and 3 are CXCR-2 inhibitors.

| | | | |
|---|---|---|---|
| 1 | AZD5122 | 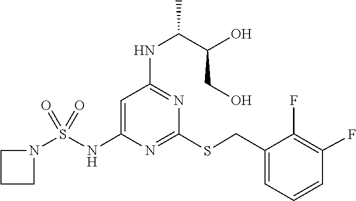 [SMILES: C[C@@H](Nc1cc(NS(=O)(=O)N2CCC2)nc(SCc2cccc(F)c2F)n1)[C@@H](O)CO] | N-(2-((2,3-difluorobenzyl)thio)-6- (((2R,3R)-3,4-dihydroxybutan-2- yl)amino)pyrimidin-4-yl)azetidine-1- sulfonamide |
| 2 | AZD8309 | 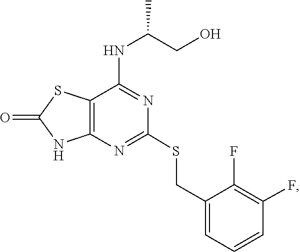 [SMILES: C[C@H](CO)Nc1nc(SCc2cccc(F)c2F)nc2[nH]c(=O)sc12] | (R)-5-((2,3-difluorobenzyl)thio)-7- ((1-hydroxypropan-2- yl)amino)thiazolo[4,5-d]pyrimidin- 2(3H)-one |
| 3 | RIST4721 | 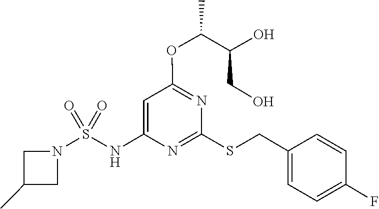 [SMILES: CC1CN(S(=O)(=O)Nc2cc(O[C@H](C)[C@@H](O)CO)nc(SCc3ccc(F)cc3)n2)C1] | N-(6-(((2R,3S)-3,4-dihydroxybutan-2- yl)oxy)-2-((4- fluorobenzyl)thio)pyrimidin-4-yl)-3- methylazetidine-1-sulfonamide |

In some embodiments, the CXCR-2 inhibitor is Compound 3, or a pharmaceutically acceptable salt thereof. Compound 3 (N-(6-(((2R,3S)-3,4-dihydroxybutan-2-yl)oxy)-2-((4-fluorobenzyl)thio)pyrimidin-4-yl)-3-methylazetidine-1-sulfonamide) is a pyrimidinyl sulphonamide and is useful as a modulator of chemokine receptors. WO 2004/011443 describes pyrimidinyl sulphonamide derivatives for use as modulators of chemokine receptors. The preparation of compound 3, along with several distinct crystalline forms, is described in WO 2013/008002.

Examples of other CXCR-2 inhibitors include but are not limited to, elubrixin, danirixin, navarixin, reparixin, ladarixin, and meraxin.

Examples of other CXCR-2 inhibitors include but are not limited to, LY-3041658, DF-1970, DF-2162, DF-2755A, CCX-872, and MGTA-M100.

Additional examples of other CXCR-2 inhibitors include, but are not limited to, the compounds in the following table:

| | | |
|---|---|---|
| AZ- 10397767 | 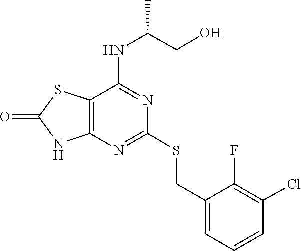 [SMILES: C[C@H](CO)Nc1nc(SCc2cccc(Cl)c2F)nc2[nH]c(=O)sc12] | (R)-5-((3-chloro-2- fluorobenzyl)thio)-7-((1- hydroxypropan-2- yl)amino)thiazolo[4,5-d] pyrimidin-2(3H)-one |
| SX-682 | 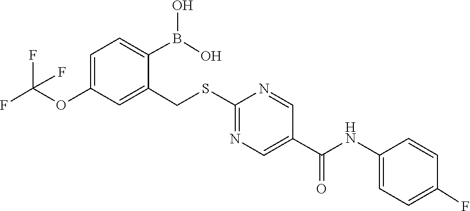 [SMILES: O=C(Nc1ccc(F)cc1)c1cnc(SCc2cc(OC(F)(F)F)ccc2B(O)O)nc1] | (2-(((5-((4- fluorophenyl)carbamoyl) pyrimidin-2-yl)thio)methyl)-4- (trifluoromethoxy)phenyl) boronic acid |
| SX-576 | 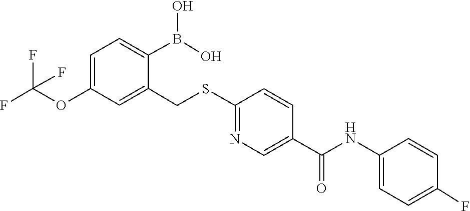 [SMILES: O=C(Nc1ccc(F)cc1)c1ccc(SCc2cc(OC(F)(F)F)ccc2B(O)O)nc1] | (2-(((5-((4- fluorophenyl)carbamoyl) pyridin-2-yl)thio)methyl)-4- (trifluoromethoxy)phenyl) boronic acid |
| SB- 265610 | 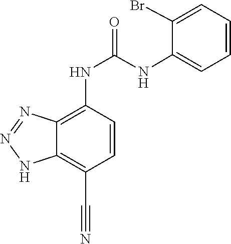 [SMILES: N#Cc1ccc(NC(=O)Nc2ccccc2Br)c2nn[nH]c12] | 1-(2-bromophenyl)-3-(7-cyano- 1H-benzo[d][1,2,3]triazol-4- yl)urea |
| SB- 332235 | 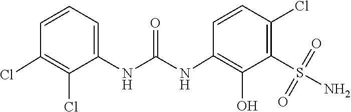 [SMILES: NS(=O)(=O)c1c(Cl)ccc(NC(=O)Nc2cccc(Cl)c2Cl)c1O] | 6-chloro-3-(3-(2,3- dichlorophenyl)ureido)-2- hydroxybenzenesulfonamide |
| SB- 656933 | 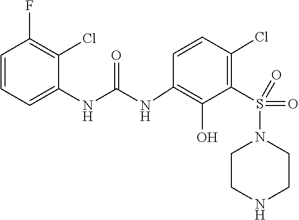 [SMILES: O=C(Nc1ccc(Cl)c(S(=O)(=O)N2CCNCC2)c1O)Nc1cccc(F)c1Cl] | 1-(4-chloro-2-hydroxy-3- (piperazin-1-ylsulfonyl)phenyl)- 3-(2-chloro-3-fluorophenyl)urea |
| GSK1325756 | 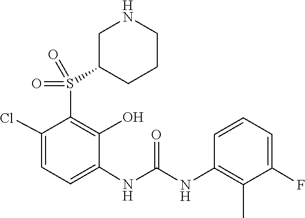 [SMILES: Cc1c(F)cccc1NC(=O)Nc1ccc(Cl)c(S(=O)(=O)[C@H]2CCCNC2)c1O] | (S)-1-(4-chloro-2-hydroxy-3- (piperidin-3-ylsulfonyl)phenyl)- 3-(3-fluoro-2-methylphenyl)urea |
| Ladarixin | 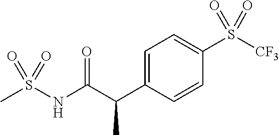 [SMILES: C[C@@H](C(=O)NS(C)(=O)=O)c1ccc(S(=O)(=O)C(F)(F)F)cc1] | (R)-N-(methylsulfonyl)-2-(4- ((trifluoromelhyl)sulfonyl) phenyl)propanamide |
| MK-7123 | 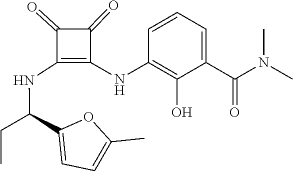 [SMILES: CC[C@@H](Nc1c(Nc2cccc(C(=O)N(C)C)c2O)c(=O)c1=O)c1ccc(C)o1] | (R)-2-hydroxy-N,N- dimethyl-3-((2-((1-(5- methylfuran-2- yl)propyl)amino)-3,4- dioxocyclobut-1-en-1- yl)amino)benzainide |

Additional examples of other CXCR-2 inhibitors include, but are not limited to, the compounds in the following table:

| | |
|---|---|
| 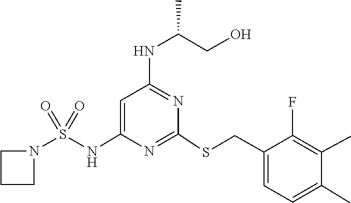 [SMILES: Cc1ccc(CSc2nc(N[C@H](C)CO)cc(NS(=O)(=O)N3CCC3)n2)c(F)c1C] | (R)-N-(2-(2-fluoro-3,4- dimethylbenzylthio)-6-(1- hydroxypropan-2- ylamino)pyrimidin-4- yl)azetidine-1-sulfonamide |
| 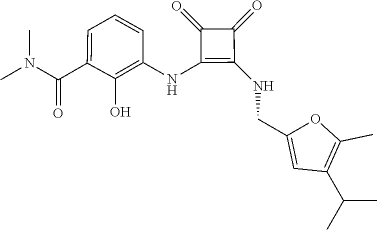 | (S)-2-hydroxy-3-(2-(1-(4- isopropyl-5-methylfuran-2- yl)propylamino)-3,4- dioxocyclobut-1-enylamino)- N,N-dimethylbenzamide |
| 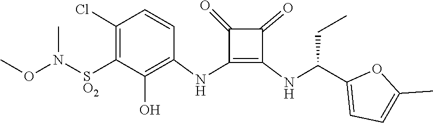 | (R)-6-chloro-2-hydroxy-N- methoxy-N-methyl-3-(2-(1-(5- methylfuran-2-yl)propylamino)- 3,4-dioxocyclobut-1- enylamino)benzenesulfonamide |
| 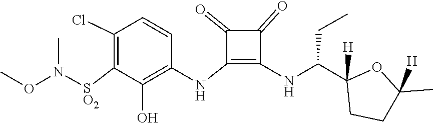 | 6-chloro-2-hydroxy-N- methoxy-N-methyl-3-(2-((R)-1- ((2R,5R)-5- methyltetrahydrofuran-2- yl)propylamino)-3,4- dioxocyclobut-1- enylamino)benzenesulfonamide |
| 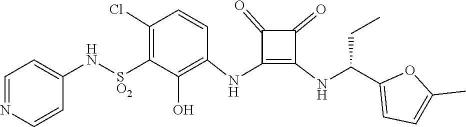 [SMILES: CC[C@@H](Nc1c(Nc2ccc(Cl)c(S(=O)(=O)Nc3ccncc3)c2O)c(=O)c1=O)c1ccc(C)o1] | (R)-6-chloro-2-hydroxy-3-(2-(1- (5-methylfuran-2- yl)propylamino)-3,4- dioxocyclobut-1-enylamino)-N- (pyridin-4- yl)benzenesulfonamide |
| 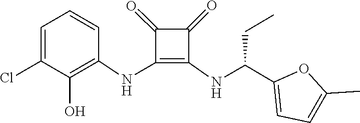 [SMILES: CC[C@@H](Nc1c(Nc2cccc(Cl)c2O)c(=O)c1=O)c1ccc(C)o1] | (R)-3-(3-chloro-2- hydroxyphenylamino)-4-(1-(5- methylfuran-2- yl)propylamino)cyclobut-3-ene- 1,2-dione |
| 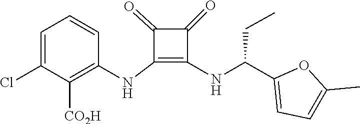 [SMILES: CC[C@@H](Nc1c(Nc2cccc(Cl)c2C(=O)O)c(=O)c1=O)c1ccc(C)o1] | (R)-2-chloro-6-(2-(1-(5- methylfuran-2-yl)propylamino)- 3,4-dioxocyclobut-1- enylamino)benzoic acid |
| 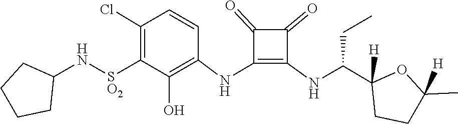 [SMILES: CC[C@@H](Nc1c(Nc2ccc(Cl)c(S(=O)(=O)NC3CCCC3)c2O)c(=O)c1=O)[C@H]1CC[C@@H](C)O1] | 6-chloro-N-cyclopentyl-2- hydroxy-3-(2-((R)-1-((2R,5R)- 5-methyltetrahydrofuran-2- yl)propylamino)-3,4- dioxocyclobut-1- enylamino)benzenesulfonamide |
| 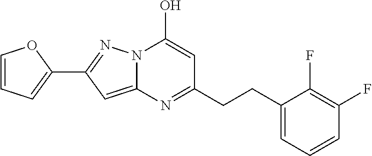 [SMILES: Oc1cc(CCc2cccc(F)c2F)nc2cc(-c3ccco3)nn12] | 5-(2,3-difluorophenethyl)-2- (furan-2-yl)pyrazolo[1,5- a]pyrimidin-7-ol |
| 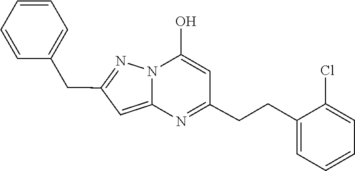 [SMILES: Oc1cc(CCc2ccccc2Cl)nc2cc(Cc3ccccc3)nn12] | 2-benzyl-5-(2- chlorophenethyl)pyrazolo[1,5- a]pyrimidin-7-ol |
| 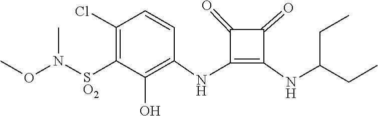 [SMILES: CCC(CC)Nc1c(Nc2ccc(Cl)c(S(=O)(=O)N(C)OC)c2O)c(=O)c1=O] | 6-chloro-3-(3,4-dioxo-2- (pentan-3-ylamino)cyclobut-1- enylamino)-2-hydroxy-N- methoxy-N- methylbenzenesulfonamide |
| 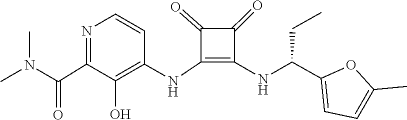 | (R)-3-hydroxy-N,N-dimethyl-4- (2-(1-(5-methylfuran-2- yl)propylamino)-3,4- dioxocyclobut-1- enylamino)picolinamide |
| 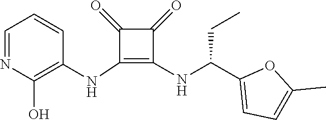 [SMILES: CC[C@@H](Nc1c(Nc2cccnc2O)c(=O)c1=O)c1ccc(C)o1] | (R)-3-(2-hydroxypyridin-3- ylamino)-4-(1-(5-methylfuran- 2-yl)propylamino)cyclobut-3- ene-1,2-dione |
| 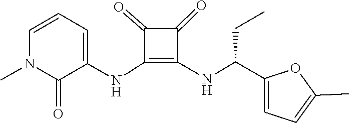 | (R)-3-(1-methyl-2-oxo-1,2- dihydropyridin-3-ylamino)-4- (1-(5-methylfuran-2- yl)propylamino)cyclobut-3-ene- 1,2-dione |
| 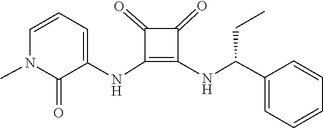 [SMILES: CC[C@@H](Nc1c(Nc2cccn(C)c2=O)c(=O)c1=O)c1ccccc1] | (R)-3-(1-methyl-2-oxo-1,2- dihydropyridin-3-ylamino)-4- (1-phenylpropylamino) cyclobut-3-ene-l,2-dione |
| 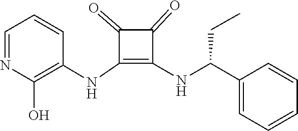 [SMILES: CC[C@@H](Nc1c(Nc2cccnc2O)c(=O)c1=O)c1ccccc1] | (R)-3-(2-hydroxypyridin-3- ylamino)-4-(1- phenylpropylamino)cyclobut-3- ene-l,2-dione |
| 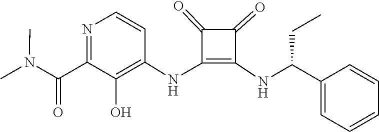 [SMILES: CC[C@@H](Nc1c(Nc2ccnc(C(=O)N(C)C)c2O)c(=O)c1=O)c1ccccc1] | (R)-4-(3,4-dioxo-2-(1- phenylpropylamino)cyclobut-1- enylamino)-3-hydroxy-N,N- dimethylpicolinamide |
| 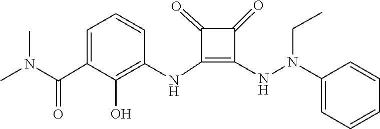 [SMILES: CCN(Nc1c(Nc2cccc(C(=O)N(C)C)c2O)c(=O)c1=O)c1ccccc1] | 3-(2-(2-ethyl-2- phenylhydrazinyl)-3,4- dioxocyclobut-1-enylamino)-2- hydroxy-N,N- dimethylbenzamide 3-(2-(2,2-diethylhydrazinyl)- |
| 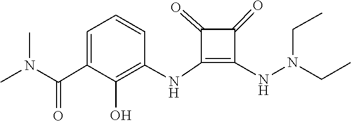 [SMILES: CCN(CC)Nc1c(Nc2cccc(C(=O)N(C)C)c2O)c(=O)c1=O] | 3,4-dioxocyclobut-1- enylamino)-2-hydroxy-N,N- dimethylbenzamide |
| 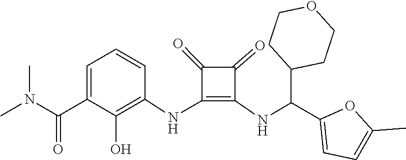 | 2-hydroxy-N,N-dimethyl-3-(2- ((5-methylfuran-2- yl)(tetrahydro-2H-pyran-4- yl)methylamino)-3,4- dioxocyclobut-1- enylamino)benzamide |
| 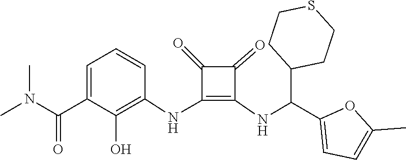 | 2-hydroxy-N,N-dimethyl-3-(2- ((5-methylfuran-2- yl)(tetrahydro-2H-thiopyran-4- yl)methylamino)-3,4- dioxocyclobut-1- enylamino)benzamide |
| 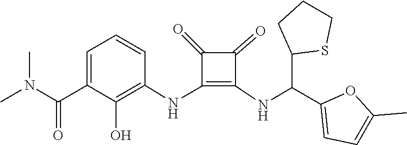 | 2-hydroxy-N,N-dimethyl-3-(2- ((5-methylfuran-2- yl)(tetrahydrothiophen-2- yl)methylamino)-3,4- dioxocyclobut-1- enylamino)benzamide |
| 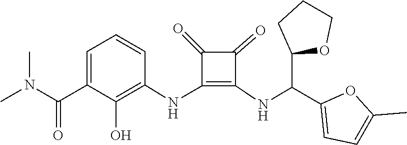 | 2-hydroxy-N,N-dimethyl-3-(2- ((5-methylfuran-2-yl)((R)- tetrahydrofuran-2- yl)methylamino)-3,4- dioxocyclobut-1- enylamino)benzamide |
| 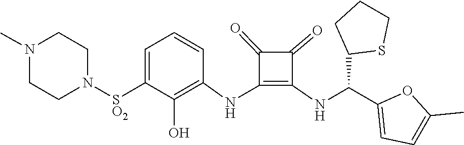 | 3-(2-hydroxy-3-(4- methylpiperazin-1- ylsulfonyl)phenylamino)-4-((S)- (5-methylfuran-2-yl)((R)- tetrahydrothiophen-2- yl)methylamino)cyclobut-3-ene- 1,2-dione |
| 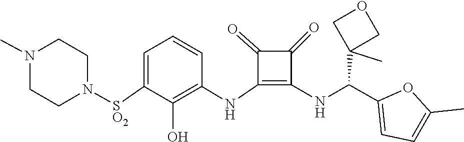 | (R)-3-(2-hydroxy-3-(4- methylpiperazin-1- ylsulfonyl)phenylamino)-4-((5- methylfuran-2-yl)(3- methyloxetan-3- yl)methylamino)cyclobut-3-ene- 1,2-dione |
| 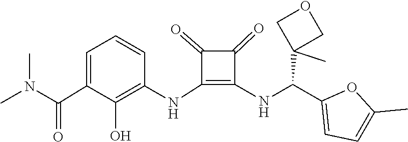 | (R)-2-hydroxy-N,N-dimethyl-3- (2-((5-methylfuran-2-yl)(3- methyloxetan-3- yl)methylamino)-3,4- dioxocyclobut-1- enylamino)benzamide |
| 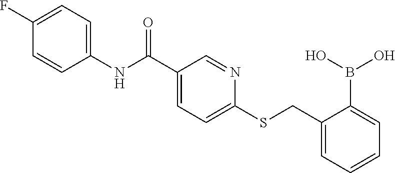 [SMILES: O=C(Nc1ccc(F)cc1)c1ccc(SCc2ccccc2B(O)O)nc1] | 2-((5-(4- fluorophenylcarbamoyl)pyridin- 2-ylthio)methyl)phenylboronic acid |
| 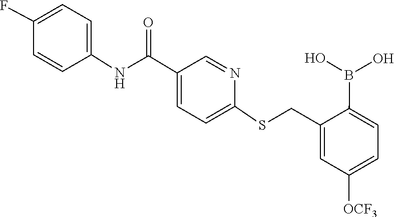 [SMILES: O=C(Nc1ccc(F)cc1)c1ccc(SCc2cc(OC(F)(F)F)ccc2B(O)O)nc1] | 2-((5-(4- fluorophenylcarbamoyl)pyridin- 2-ylthio)methyl)-4- (trifluoromethoxy) phenylboronic acid |
| 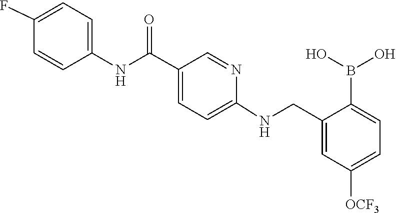 [SMILES: O=C(Nc1ccc(F)cc1)c1ccc(NCc2cc(OC(F)(F)F)ccc2B(O)O)nc1] | 2-((5-(4- fluorophenylcarbamoyl)pyridin- 2-ylamino)methyl)-4- (trifluoromethoxy) phenylboronic acid |
| 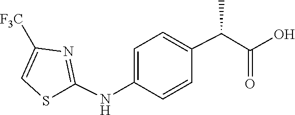 [SMILES: C[C@H](C(=O)O)c1ccc(Nc2nc(C(F)(F)F)cs2)cc1] | (S)-2-(4-(4- (trifluoromethyl)thiazol-2- ylamino)phenyl)propanoic acid |
| 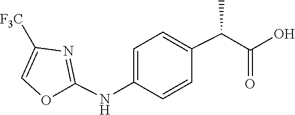 [SMILES: C[C@H](C(=O)O)c1ccc(Nc2nc(C(F)(F)F)co2)cc1] | (S)-2-(4-(4- (trifluoromethyl)oxazol-2- ylamino)phenyl)propanoic acid |
| 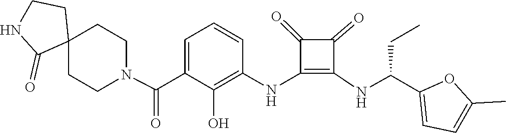 [SMILES: CC[C@@H](Nc1c(Nc2cccc(C(=O)N3CCC4(CCNC4=O)CC3)c2O)c(=O)c1=O)c1ccc(C)o1] | (R)-3-(2-hydroxy-3-(1-oxo-2,8- diazaspiro[4.5]decane-8- carbonyl)phenylamino)-4-(1-(5- methylfuran-2- yl)propylamino)cyclobut-3-ene- 1,2-dione |
| 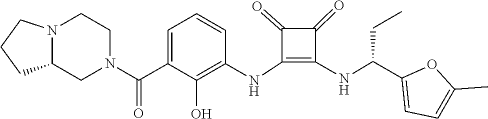 [SMILES: CC[C@@H](Nc1c(Nc2cccc(C(=O)N3CCN4CCC[C@H]4C3)c2O)c(=O)c1=O)c1ccc(C)o1] | 3-(2-hydroxy-3-((S)- octahydropyrrolo[1,2- a]pyrazine-2- carbonyl)phenylamino)-4- ((R)-1-(5-methylfuran-2- yl)propylamino)cyclobut-3- ene-1,2-dione |
| 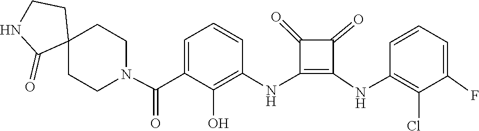 [SMILES: O=C(c1cccc(Nc2c(Nc3cccc(F)c3Cl)c(=O)c2=O)c1O)N1CCC2(CCNC2=O)CC1] | 3-(2-chloro-3- fluorophenylamino)-4-(2- hydroxy-3-(1-oxo-2,8- diazaspiro[4.5]decane-8- carbonyl)phenylamino) cyclobut-3-ene-1,2-dione |
| 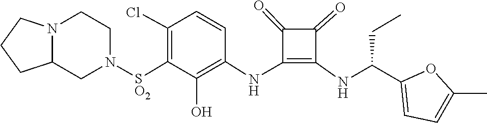 [SMILES: CC[C@@H](Nc1c(Nc2ccc(Cl)c(S(=O)(=O)N3CCN4CCCC4C3)c2O)c(=O)c1=O)c1ccc(C)o1] | 3-(4-chloro-3- (hexahydropyrrolo[1,2-a] pyrazin-2(1H)-ylsulfonyl)-2- hydroxyphenylamino)-4-((R)-1- (5-methylfuran-2- yl)propylamino)cyclobut-3-ene- 1,2-dione |
| 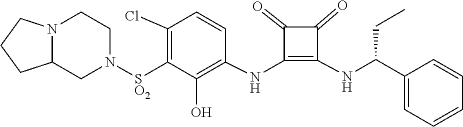 [SMILES: CC[C@@H](Nc1c(Nc2ccc(Cl)c(S(=O)(=O)N3CCN4CCCC4C3)c2O)c(=O)c1=O)c1ccccc1] | 3-(4-chloro-3- (hexahydropyrrolo[1,2- a]pyrazin-2(1H)-ylsulfonyl)-2- hydroxyphenylamino)-4-((R)-1- phenylpropylamino)cyclobut-3- ene-1,2-dione |
| 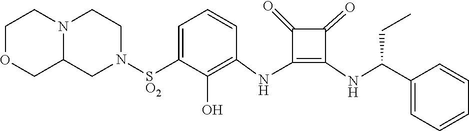 [SMILES: CC[C@@H](Nc1c(Nc2cccc(S(=O)(=O)N3CCN4CCOCC4C3)c2O)c(=O)c1=O)c1ccccc1] | 3-(3-(hexahydropyrazino[2,1- c][1,4]oxazin-8(1H)- ylsulfonyl)-2- hydroxyphenylamino)-4-((R)-1- phenylpropylamino)cyclobut-3- ene-1,2-dione |
| 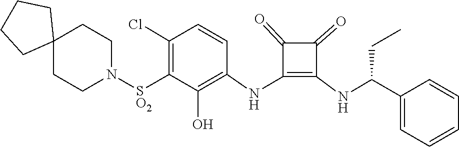 [SMILES: CC[C@@H](Nc1c(Nc2ccc(Cl)c(S(=O)(=O)N3CCC4(CCCC4)CC3)c2O)c(=O)c1=O)c1ccccc1] | 3-(3-(8-azaspiro[4.5]decan-8- ylsulfonyl)-4-chloro-2- hydroxyphenylamino)-4-((R)-1- phenylpropylamino)cyclobut-3- ene-1,2-dione |
| 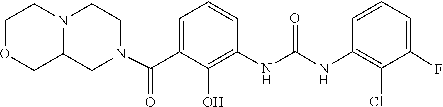 [SMILES: O=C(Nc1cccc(C(=O)N2CCN3CCOCC3C2)c1O)Nc1cccc(F)c1Cl] | 1-(2-chloro-3-fluorophenyl)-3- (2-hydroxy-3- (octahydropyrazino[2,1- c][1,4]oxazine-8- carbonyl)phenyl)urea |
| 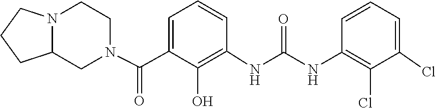 [SMILES: O=C(Nc1cccc(C(=O)N2CCN3CCCC3C2)c1O)Nc1cccc(Cl)c1Cl] | 1-(2,3-dichiorophenyl)-3-(2- hydroxy-3-(octahydropyrrolo [1,2-a]pyrazine-2- carbonyl)phenyl)urea |

### CXCR-2 Inhibitor Dosing

The amount of CXCR-2 inhibitor administered will firstly be dependent on the mammal being treated. In some embodiments, the daily dosage will normally be determined by the prescribing physician with the dosage generally varying according to the age, sex, diet, weight, general health and response of the individual patient, the severity of the patient's symptoms, the precise indication or condition being treated, the severity of the indication or condition being treated, time of administration, route of administration, drug absorption, the disposition of the composition, rate of excretion, drug combination, and the discretion of the prescribing physician. Also, the route of administration varies depending on the condition and its severity. The pharmaceutical composition is, in some embodiments, in unit dosage form. In such form, the preparation is subdivided into unit doses containing appropriate quantities of the active component, e.g., an effective amount to achieve the desired purpose. Determination of the proper dosage for a particular situation is within the skill of the art. In some embodiments, the total daily dosage is divided and administered in portions during the day if desired. The amount and frequency of administration will be regulated according to the judgment of the attending clinician physician considering such factors as described above. Thus, the amount of pharmaceutical composition to be administered is variable depending upon the circumstances. In some instances, dosage levels below the lower limit of the aforesaid range are more than adequate, while in other cases still larger doses are employed without causing any harmful side effect, e.g. by dividing such larger doses into several small doses for administration throughout the day. In combinational applications in which the compound is not the sole therapy, it is possible to administer lesser amounts of compound and still have therapeutic or prophylactic effect.

In some embodiments, the methods comprise administering between about 1 mg/day and about 1000 mg/day of a CXCR-2 inhibitor. In some embodiments, the methods comprise administering between about 10 mg/day and about 1000 mg/day of a CXCR-2 inhibitor. In some embodiments, the methods comprise administering between about 10 mg/day and about 500 mg/day of a CXCR-2 inhibitor. In some embodiments, the methods comprise administering between about 100 mg/day and about 500 mg/day of a CXCR-2 inhibitor. In some embodiments, the methods comprise administering between about 200 mg/day and about 500 mg/day of a CXCR-2 inhibitor. In some embodiments, the methods comprise administering between about 300 mg/day and about 500 mg/day of a CXCR-2 inhibitor. In some embodiments, the methods comprise administering between about 300 mg/day and about 400 mg/day of a CXCR-2 inhibitor.

In some embodiments, the methods comprise administering about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 105 mg/day, about 110 mg/day, about 115 mg/day, about 120 mg/day, about 125 mg/day, about 130 mg/day, about 135 mg/day, about 140 mg/day, about 145 mg/day, about 150 mg/day, about 155 mg/day, about 160 mg/day, about 165 mg/day, about 170 mg/day, about 175 mg/day, about 180 mg/day, about 185 mg/day, about 190 mg/day, about 195 mg/day, about 200 mg/day, about 205 mg/day, about 210 mg/day, about 215 mg/day, about 220 mg/day, about 225 mg/day, about 230 mg/day, about 235 mg/day, about 240 mg/day, about 245 mg/day, about 250 mg/day, about 255 mg/day, about 260 mg/day, about 265 mg/day, about 270 mg/day, about 275 mg/day, about 280 mg/day, about 285 mg/day, about 290 mg/day, about 295 mg/day, about 300 mg/day, about 305 mg/day, about 310 mg/day, about 315 mg/day, about 320 mg/day, about 325 mg/day, about 330 mg/day, about 335 mg/day, about 340 mg/day, about 345 mg/day, about 350 mg/day, about 355 mg/day, about 360 mg/day, about 365 mg/day, about 370 mg/day, about 375 mg/day, about 380 mg/day, about 385 mg/day, about 390 mg/day, about 395 mg/day, about 400 mg/day, about 405 mg/day, about 410 mg/day, about 415 mg/day, about 420 mg/day, about 425 mg/day, about 430 mg/day, about 435 mg/day, about 440 mg/day, about 445 mg/day, about 450 mg/day, about 455 mg/day, about 460 mg/day, about 465 mg/day, about 470 mg/day, about 475 mg/day, about 480 mg/day, about 485 mg/day, about 490 mg/day, about 495 mg/day, or about 500 mg/day of a CXCR-2 inhibitor. In some embodiments, the methods comprise administering about 30 mg/day of a CXCR-2 inhibitor. In some embodiments, the methods comprise administering about 35 mg/day of a CXCR-2 inhibitor. In some embodiments, the methods comprise administering about 50 mg/day of a CXCR-2 inhibitor. In some embodiments, the methods comprise administering about 100 mg/day of a CXCR-2 inhibitor. In some embodiments, the methods comprise administering about 150 mg/day of a CXCR-2 inhibitor. In some embodiments, the methods comprise administering about 200 mg/day of a CXCR-2 inhibitor. In some embodiments, the methods comprise administering about 250 mg/day of a CXCR-2 inhibitor. In some embodiments, the methods comprise administering about 300 mg/day of a CXCR-2 inhibitor. In some embodiments, the methods comprise administering about 350 mg/day of a CXCR-2 inhibitor. In some embodiments, the methods comprise administering about 400 mg/day of a CXCR-2 inhibitor.

### Methods

Described herein are methods of treating a subject suffering from or at risk of suffering from Familial Mediterranean Fever (FMF), which comprises administering to the subject a therapeutically effective amount of a CXCR-2 inhibitor, or a pharmaceutically acceptable salt thereof.

Also described herein are methods of preventing flare-ups in a subject having been diagnosed with Familial Mediterranean Fever (FMF), which comprises administering to the patient a therapeutically effective amount of a CXCR-2 inhibitor, or a pharmaceutically acceptable salt thereof.

### Familial Mediterranean Fever (FMF)

Familial Mediterranean fever (FMF), also known as familial paroxysmal polyserositis, periodic peritonitis, recurrent polyserositis, benign paroxysmal peritonitis, Reimann periodic disease or Reimann syndrome, Siegal-Cattan-Mamou disease, and Wolff periodic disease, is a hereditary inflammatory disorder. FMF is an autoinflammatory disease caused by mutations in Mediterranean fever gene, which encodes a 781-amino acid protein called pyrin. While all ethnic groups are susceptible to FMF, it commonly occurs in people of Mediterranean origin-including Sephardic Jews, Mizrahi Jews, Ashkenazi Jews, Armenians, Azerbaijanis, Arabs, Kurds, Greeks, Turks, Iranians and Italians.

Pyrin acts as an intranuclear regulator of transcription of the peptides involved in inflammation. Pyrin regulates the inflammatory response by blocking intracellular signal pathways via nuclear factor kβ (NF-kβ) or caspase 1. Patients with FMF have an absence of pyrin function due to mutated MEFV which leads to over secretion of inflammatory cytokines and results in a hyperinflammatory response characterized by abundant neutrophilic infiltration into peritoneal, pleural and joint spaces. Acute FMF attacks develop because of neutrophil recruitment and activation at the serosal and synovial surfaces.

Uncontrolled FMF attacks leads to secondary amyloidosis, which is the primary cause of morbidity and mortality (due to cardiac amyloidosis and renal failure).

Colchicine affects the mechanics of neutrophils and, thereby, motility in confined spaces, which is crucial during extravasation of neutrophils in response to inflammatory stimuli. CXCR-2 Inhibitors block the trafficking of neutrophils from the bone marrow to the specific site of inflammation. In some embodiments, for patients not responsive to colchicine therapy alone, affecting both recruitment and extravasation of neutrophils decreases the number of attacks (flare-ups) in patients with FMF.

FMF is characterized by sporadic attacks or flare-ups. Patients who are not controlled will have at least 1 flare-up per month.

There are seven types of attacks (flare-ups). Ninety percent of all patients have their first attack before they are 18 years old. All develop over 2-4 hours and last anywhere from 6 hours to 4 days. Most attacks involve fever.

1. Abdominal attacks, featuring abdominal pain, affect the whole abdomen with all signs of peritonitis (inflammation of abdominal lining), and acute abdominal pain like appendicitis. Incomplete attacks, with local tenderness and normal blood tests, have been reported.
2. Joint attacks mainly occur in large joints, especially in the legs. Usually, only one joint is affected. 75% of all FMF patients experience joint attacks.
3. Chest attacks include pleuritis (inflammation of the pleura) and pericarditis (inflammation of the pericardium). Pleuritis occurs in 40% of patients and makes it difficult to breathe or lie flat, but pericarditis is rare.
4. Scrotal attacks due to inflammation of the tunica vaginalis.

### 5. Myalgia.

6. Erysipeloid (a skin reaction on the legs that can mimic cellulitis).
7. Fever without any of the other symptoms listed above (25%). Fever may be the only symptom during the childhood. It may vary from a mild fever to 38-40° C.

In some embodiments, the subject having diagnosed with FMF will experience FMF flare-ups. In some embodiments, the FMF flare-ups comprises fever, abdominal pain, joint pain, pleuritis, pericarditis, or any combinations thereof.

In some embodiments, the FMF flare-up is an acute FMF flare-up. In some embodiments, the subject presents with a C-reactive protein (CRP) level of greater than 10 mg/L. In some embodiments, the subject presents with a C-reactive protein (CRP) level of greater than 20 mg/L. In some embodiments, the subject presents with a C-reactive protein (CRP) level of greater than 30 mg/L. In some embodiments, the subject presents with a C-reactive protein (CRP) level of greater than 40 mg/L. In some embodiments, the subject presents with a C-reactive protein (CRP) level of greater than 50 mg/L. In some embodiments, the subject presents with a C-reactive protein (CRP) level of greater than 60 mg/L. In some embodiments, the subject presents with a C-reactive protein (CRP) level of greater than 70 mg/L. In some embodiments, the subject presents with a C-reactive protein (CRP) level of greater than 80 mg/L. In some embodiments, the subject presents with a C-reactive protein (CRP) level of greater than 90 mg/L. In some embodiments, the subject presents with a C-reactive protein (CRP) level of greater than 100 mg/L. In some embodiments, the subject presents with a C-reactive protein (CRP) level of from about 10 mg/L to about 1,000 mg/L. In some embodiments, the subject presents with a C-reactive protein (CRP) level of from about 20 mg/L to about 1,000 mg/L. In some embodiments, the subject presents with a C-reactive protein (CRP) level of from about 30 mg/L to about 1,000 mg/L. In some embodiments, the subject presents with a C-reactive protein (CRP) level of from about 40 mg/L to about 1,000 mg/L. In some embodiments, the subject presents with a C-reactive protein (CRP) level of from about 50 mg/L to about 1,000 mg/L. In some embodiments, the subject presents with a C-reactive protein (CRP) level of from about 60 mg/L to about 1,000 mg/L. In some embodiments, the subject presents with a C-reactive protein (CRP) level of from about 70 mg/L to about 1,000 mg/L. In some embodiments, the subject presents with a C-reactive protein (CRP) level of from about 80 mg/L to about 1,000 mg/L. In some embodiments, the subject presents with a C-reactive protein (CRP) level of from about 90 mg/L to about 1,000 mg/L. In some embodiments, the subject presents with a C-reactive protein (CRP) level of from about 100 mg/L to about 1,000 mg/L.

### Combination

Described herein are combination therapies wherein any one of the CXCR-2 inhibitors disclosed herein, or pharmaceutically acceptable salts thereof, is administered concurrently or sequentially with an additional therapy and/or an agent for the treatment of Familial Mediterranean Fever (FMF). In some embodiments, a CXCR-2 inhibitor, or pharmaceutically acceptable salt thereof, is administered concurrently or sequentially with an additional agent for the prevention of flare-ups in a subject having been diagnosed with FMF. In some embodiments, the CXCR-2 inhibitor, or pharmaceutically acceptable salts thereof, is administered concurrently or sequentially with colchicine. In some embodiments, the CXCR-2 inhibitor is compound 1, 2, or 3, or the pharmaceutically acceptable salt thereof. In specific embodiments, the CXCR-2 inhibitor is compound 3 or the pharmaceutically acceptable salt thereof.

Also described herein are methods for treating acute FMF flare-ups by concomitantly or sequentially administering to a subject in need thereof a combination of (i) Colchicine, or a pharmaceutically acceptable salt thereof, and (ii) a CXCR-2 inhibitor. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is N-(2-((2,3-difluorobenzyl)thio)-6-(((2R,3R)-3,4-dihydroxybutan-2-yl)amino)pyrimidin-4-yl)azetidine-1-sulfonamide (compound 1), or a pharmaceutically acceptable salt thereof. In some embodiments, the CXCR-2 inhibitor is (R)-5-((2,3-difluorobenzyl)thio)-7-((1-hydroxypropan-2-yl)amino)thiazolo[4,5-d]pyrimidin-2(3H)-one (compound 2), or a pharmaceutically acceptable salt thereof. In some embodiments, the CXCR-2 inhibitor is N-(6-(((2R,3 S)-3,4-dihydroxybutan-2-yl)oxy)-2-((4-fluorobenzyl)thio)pyrimidin-4-yl)-3-methylazetidine-1-sulfonamide (compound 3) or a pharmaceutically acceptable salt thereof.

Also described herein are methods for preventing FMF flare-ups by concomitantly or sequentially administering to a subject in need thereof a combination of (i) Colchicine, or a pharmaceutically acceptable salt thereof, and (ii) a CXCR-2 inhibitor. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is N-(2-((2,3-difluorobenzyl)thio)-6-(((2R,3R)-3,4-dihydroxybutan-2-yl)amino)pyrimidin-4-yl)azetidine-1-sulfonamide (compound 1), or a pharmaceutically acceptable salt thereof. In some embodiments, the CXCR-2 inhibitor is (R)-5-((2,3-difluorobenzyl)thio)-7-((1-hydroxypropan-2-yl)amino)thiazolo[4,5-d]pyrimidin-2(3H)-one (compound 2), or a pharmaceutically acceptable salt thereof. In some embodiments, the CXCR-2 inhibitor is N-(6-(((2R,3 S)-3,4-dihydroxybutan-2-yl)oxy)-2-((4-fluorobenzyl)thio)pyrimidin-4-yl)-3-methylazetidine-1-sulfonamide (compound 3) or a pharmaceutically acceptable salt thereof.

Also described herein are methods for decreasing the severity of FMF flare-up by concomitantly or sequentially administering to a subject in need thereof a combination of (i) Colchicine, or a pharmaceutically acceptable salt thereof; and (ii) a CXCR-2 inhibitor. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is N-(2-((2,3-difluorobenzyl)thio)-6-(((2R,3R)-3,4-dihydroxybutan-2-yl)amino)pyrimidin-4-yl)azetidine-1-sulfonamide (compound 1), or a pharmaceutically acceptable salt thereof. In some embodiments, the CXCR-2 inhibitor is (R)-5-((2,3-difluorobenzyl)thio)-7-((1-hydroxypropan-2-yl)amino)thiazolo[4,5-d]pyrimidin-2(3H)-one (compound 2), or a pharmaceutically acceptable salt thereof. In some embodiments, the CXCR-2 inhibitor is N-(6-(((2R,3 S)-3,4-dihydroxybutan-2-yl)oxy)-2-((4-fluorobenzyl)thio)pyrimidin-4-yl)-3-methylazetidine-1-sulfonamide (compound 3) or a pharmaceutically acceptable salt thereof.

In some embodiments, the severity of the FMF flare-ups is decreased as compared to a subject being administered Colchicine alone. In some embodiments, the severity of the FMF flare-ups is decreased as compared to a subject being administered the CXCR-2 inhibitor alone. In some embodiments, the severity of the FMF flare-ups is decreased as compared to a subject receiving no therapy. In some embodiments, the severity of the flare-ups is decreased by at least about 10%. In some embodiments, the severity of the flare-ups is decreased by at least about 20%. In some embodiments, the severity of the flare-ups is decreased by at least about 30%. In some embodiments, the severity of the flare-ups is decreased by at least about 40%. In some embodiments, the severity of the flare-ups is decreased by at least about 50%. In some embodiments, the severity of the flare-ups is decreased by at least about 60%. In some embodiments, the severity of the flare-ups is decreased by at least about 70%. In some embodiments, the severity of the flare-ups is decreased by at least about 80%. In some embodiments, the severity of the flare-ups is decreased by at least about 90%. In some embodiments, the severity of the flare-ups is decreased by at least about 99%.

Also described herein are methods for decreasing the occurrence of FMF flare-up by concomitantly or sequentially administering to a subject in need thereof a combination of (i) Colchicine, or a pharmaceutically acceptable salt thereof; and (ii) a CXCR-2 inhibitor. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is N-(2-((2,3-difluorobenzyl)thio)-6-(((2R,3R)-3,4-dihydroxybutan-2-yl)amino)pyrimidin-4-yl)azetidine-1-sulfonamide (compound 1), or a pharmaceutically acceptable salt thereof. In some embodiments, the CXCR-2 inhibitor is (R)-5-((2,3-difluorobenzyl)thio)-7-((1-hydroxypropan-2-yl)amino)thiazolo[4,5-d]pyrimidin-2(3H)-one (compound 2), or a pharmaceutically acceptable salt thereof. In some embodiments, the CXCR-2 inhibitor is N-(6-(((2R,3 S)-3,4-dihydroxybutan-2-yl)oxy)-2-((4-fluorobenzyl)thio)pyrimidin-4-yl)-3-methylazetidine-1-sulfonamide (compound 3) or a pharmaceutically acceptable salt thereof.

In some embodiments, the occurrence of the FMF flare-ups is decreased as compared to a subject being administered Colchicine alone. In some embodiments, the occurrence of the FMF flare-ups is decreased as compared to a subject being administered the CXCR-2 inhibitor alone. In some embodiments, the occurrence of the FMF flare-ups is decreased as compared to a subject receiving no therapy. In some embodiments, the occurrence of the flare-ups is decreased by at least about 10%. In some embodiments, the occurrence of the flare-ups is decreased by at least about 20%. In some embodiments, the occurrence of the flare-ups is decreased by at least about 30%. In some embodiments, the occurrence of the flare-ups is decreased by at least about 40%. In some embodiments, the occurrence of the flare-ups is decreased by at least about 50%. In some embodiments, the occurrence of the flare-ups is decreased by at least about 60%. In some embodiments, the occurrence of the flare-ups is decreased by at least about 70%. In some embodiments, the occurrence of the flare-ups is decreased by at least about 80%. In some embodiments, the occurrence of the flare-ups is decreased by at least about 90%. In some embodiments, the occurrence of the flare-ups is decreased by at least about 99%. In some embodiments, the occurrence of the flare-ups is less than 5 times a month. In some embodiments, the occurrence of the flare-ups is less than 4 times a month. In some embodiments, the occurrence of the flare-ups is less than 3 times a month. In some embodiments, the occurrence of the flare-ups is less than twice a month. In some embodiments, the occurrence of the flare-ups is less than once a month. In some embodiments, the occurrence of the flare-ups is every other month. In some embodiments, the occurrence of the flare-ups is every three months. In some embodiments, the occurrence of the flare-ups is every four months.

Also described herein are methods for improving the therapeutic index of colchicine in a subject by concomitantly or sequentially administering to a subject in need thereof a combination of (i) Colchicine, or a pharmaceutically acceptable salt thereof; and (ii) a CXCR-2 inhibitor. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is N-(2-((2,3-difluorobenzyl)thio)-6-(((2R,3R)-3,4-dihydroxybutan-2-yl)amino)pyrimidin-4-yl)azetidine-1-sulfonamide (compound 1), or a pharmaceutically acceptable salt thereof. In some embodiments, the CXCR-2 inhibitor is (R)-5-((2,3-difluorobenzyl)thio)-7-((1-hydroxypropan-2-yl)amino)thiazolo[4,5-d]pyrimidin-2(3H)-one (compound 2), or a pharmaceutically acceptable salt thereof. In some embodiments, the CXCR-2 inhibitor is N-(6-(((2R,3S)-3,4-dihydroxybutan-2-yl)oxy)-2-((4-fluorobenzyl)thio)pyrimidin-4-yl)-3-methylazetidine-1-sulfonamide (compound 3) or a pharmaceutically acceptable salt thereof.

Also described herein are methods for the prophylaxis and treatment of FMF flare-ups in a subject by concomitantly or sequentially administering to a subject in need thereof a combination of (i) Colchicine, or a pharmaceutically acceptable salt thereof; and (ii) a CXCR-2 inhibitor. In some embodiments, the subject is an adult. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is N-(2-((2,3-difluorobenzyl)thio)-6-(((2R,3R)-3,4-dihydroxybutan-2-yl)amino)pyrimidin-4-yl)azetidine-1-sulfonamide (compound 1), or a pharmaceutically acceptable salt thereof. In some embodiments, the CXCR-2 inhibitor is (R)-5-((2,3-difluorobenzyl)thio)-7-((1-hydroxypropan-2-yl)amino)thiazolo[4,5-d]pyrimidin-2(3H)-one (compound 2), or a pharmaceutically acceptable salt thereof. In some embodiments, the CXCR-2 inhibitor is N-(6-(((2R,3 S)-3,4-dihydroxybutan-2-yl)oxy)-2-((4-fluorobenzyl)thio)pyrimidin-4-yl)-3-methylazetidine-1-sulfonamide (compound 3) or a pharmaceutically acceptable salt thereof.

Also described herein are methods for preventing or reducing the incidence of amyloidosis by concomitantly or sequentially administering to a subject in need thereof a combination of (i) Colchicine, or a pharmaceutically acceptable salt thereof; and (ii) a CXCR-2 inhibitor. In some embodiments, the combination is a synergistic combination. In some embodiments, the CXCR-2 inhibitor is one of the compounds disclosed herein, or a pharmaceutically acceptable salt thereof. In some embodiments, the CXCR-2 inhibitor is N-(2-((2,3-difluorobenzyl)thio)-6-(((2R,3R)-3,4-dihydroxybutan-2-yl)amino)pyrimidin-4-yl)azetidine-1-sulfonamide (compound 1), or a pharmaceutically acceptable salt thereof. In some embodiments, the CXCR-2 inhibitor is (R)-5-((2,3-difluorobenzyl)thio)-7-((1-hydroxypropan-2-yl)amino)thiazolo[4,5-d]pyrimidin-2(3H)-one (compound 2), or a pharmaceutically acceptable salt thereof. In some embodiments, the CXCR-2 inhibitor is N-(6-(((2R,3S)-3,4-dihydroxybutan-2-yl)oxy)-2-((4-fluorobenzyl)thio)pyrimidin-4-yl)-3-methylazetidine-1-sulfonamide (compound 3) or a pharmaceutically acceptable salt thereof.

### Synergy

In some embodiments, administration of a combination of colchicine and a CXCR-2 inhibitor provides a synergistic effect. As used herein, the terms “synergy,” “synergistically,” “synergistic” or other grammatical equivalents thereof refer to a combination of therapies (e.g., colchicine and a CXCR-2 inhibitor) that is more effective than the expected additive effects of any two or more single therapies. For example, a synergistic effect of a combination of therapies permits the use of lower dosages of one or more of the therapies and/or less frequent administration of said therapies to a subject. The ability to utilize lower dosages of therapies and/or to administer the therapies less frequently reduces the toxicity associated with the administration of the therapies to a subject without reducing the efficacy of said therapies in the prevention, management, treatment, or amelioration of a given disease, such as FMF. In addition, a synergistic effect can result in improved efficacy of therapies in the prevention, management, treatment, or amelioration of a given disease, such as FMF. Synergistic effects of a combination of therapies may avoid or reduce adverse or unwanted side effects associated with the use of any single therapy. The “synergy,” “synergism,” or “synergistic” effect of a combination may be determined herein by the methods of Chou et al., and/or Clarke et al. See Ting-Chao Chou, Theoretical Basis, Experimental Design, and Computerized Simulation of Synergism and Antagonism in Drug Combination Studies, Pharmacol Rev 58:621-681 (2006), and Clarke et al., Issues in experimental design and endpoint analysis in the study of experimental cytotoxic agents in vivo in breast cancer and other models, Breast Cancer Research and Treatment 46:255-278 (1997), which are both incorporated by reference for the methods of determining the “synergy,” synergism,” or “synergistic” effect of a combination.

In some embodiments, the co-administration of the CXCR-2 inhibitor, such as compound 3, or pharmaceutically acceptable salts thereof, results in the need for a smaller dose of a second active agent (e.g., colchicine). In some embodiments, the co-administration of a second active agent (e.g., colchicine) and the CXCR-2 inhibitor, such as compound 3, or pharmaceutically acceptable salts thereof, results in the need for a smaller dose of the CXCR-2 inhibitor, such as compound 3, or pharmaceutically acceptable salts thereof, to treat a disease or disorder. In some embodiments, the co-administration of the CXCR-2 inhibitor, such as compound 3, or pharmaceutically acceptable salts thereof, results in the need for a smaller dose of colchicine to treat or prevent an FMF attack. In some embodiments, the co-administration of the CXCR-2 inhibitor, such as compound 3, or pharmaceutically acceptable salts thereof, results in the need for a smaller dose of colchicine to treat or prevent an FMF attack. In some embodiments, the smaller dose of colchicine and/CXCR-2 inhibitor is a sub-therapeutically effective amount.

It is difficult to predict the effect of many combination therapies. For example, some drugs interact with each other to reduce therapeutic effectiveness or cause undesired side-effects. These drugs are typically categorized as having an antagonistic effect. Other drug combinations manifest their therapeutic effectiveness as the sum of individual drugs. These combinations are categorized as having an additive effect. Still other drug combinations result in a therapeutic index that is greater than the sum of individual drugs. These are categorized as having a synergistic effect.

Combination therapies having a synergistic effect are highly desirable for many reasons. In some instances, each component in the synergistic combination therapy is used in an amount lower than the therapeutic amount of each individual drug in monotherapy (i.e., single drug administration). Moreover, the risk and/or the severity of side-effects can be reduced significantly by reducing the amount of each drug. Furthermore, combination therapy may significantly increase the overall effectiveness of treatment.

Synergistic actions of combination therapy are particularly useful in treatments where the side-effects are extreme or severe and/or where the efficacy of monotherapy is less than desirable.

### Additional Combination Agent

In some embodiments, the methods disclosed herein further comprise administering an additional therapeutic agent (a third agent). In some embodiments, the third agent is an IL-1 agent. In some embodiments, the third agent is Canakinumab. Canakinumab is a fully human monoclonal anti-IL-1β antibody developed to treat various inflammatory disorders. It binds to IL-1β in circulation and blocks its interaction with the IL-1 receptor by neutralizing its activity, but does not interfere with the IL-1 signal. In some embodiments, the third agent is an NSAID (such as diclofenac). In some embodiments, the third agent is anakinra. In some embodiments, the third agent is dapsone.

In some embodiments, the third agent is an IL-17 inhibitor. Examples IL-17 inhibitors include but are not limited to, Secukinumab, Brodalumab, and Ixekizumab.

In some embodiments, the third agent is an IL-23 inhibitor. Examples IL-23 inhibitors include but are not limited to, Guselkumab, Ustekinumab, Tildrakizumab, and Riskankizumab.

In some embodiments, the third agent is an IL-36 inhibitor. Examples IL-36 inhibitors include but are not limited to, BI 655130 and ANB019.

In some embodiments, the third agent is NLRP3 inflammasome inhibitor. Examples NLRP3 inflammasome inhibitors include but are not limited to, Glyburide, CAS16673-34-0, JC124, FC11A-2, Parthenolide, VX-740, VX-765, Bay 11-7082, BHB NLRP3, MCC950, MNS NLRP3, CY-09, Tranilast, OLT1177, and Oridonin.

In some embodiments, the third agent is caspase-1 inhibitor.

### Pharmaceutical Compositions

The compound, compound forms and compositions described herein are administered either alone, or in combination with, pharmaceutically acceptable adjuvants, carriers, excipients, or diluents in a pharmaceutical composition, according to standard pharmaceutical practice.

Described herein in some embodiments are pharmaceutical compositions comprising a CXCR-2 inhibitor, such as any one of the compounds described herein, or the pharmaceutically acceptable salt thereof in association with a pharmaceutically acceptable adjuvant, diluent or carrier. In some embodiments, the CXCR-2 inhibitor is N-(2-((2,3-difluorobenzyl)thio)-6-(((2R,3R)-3,4-dihydroxybutan-2-yl)amino)pyrimidin-4-yl)azetidine-1-sulfonamide (compound 1), or a pharmaceutically acceptable salt thereof. In some embodiments, the CXCR-2 inhibitor is (R)-5-((2,3-difluorobenzyl)thio)-7-((1-hydroxypropan-2-yl)amino)thiazolo[4,5-d]pyrimidin-2(3H)-one (compound 2), or a pharmaceutically acceptable salt thereof. In some embodiments, the CXCR-2 inhibitor is N-(6-(((2R,3 S)-3,4-dihydroxybutan-2-yl)oxy)-2-((4-fluorobenzyl)thio)pyrimidin-4-yl)-3-methylazetidine-1-sulfonamide (compound 3), or a pharmaceutically acceptable salt thereof. In some embodiments, the pharmaceutical compositions further comprise colchicine.

Also described herein in some embodiments are pharmaceutical compositions comprising colchicine and a CXCR-2 inhibitor in association with a pharmaceutically acceptable adjuvant, diluent or carrier. Also disclosed herein are pharmaceutical compositions comprising a therapeutically-effective amount of Colchicine, and a therapeutically-effective amount of a CXCR-2 inhibitor. Also disclosed herein are pharmaceutical compositions comprising a sub-therapeutically-effective amount of Colchicine, and a sub-therapeutically-effective amount of a CXCR-2 inhibitor. In some embodiments, the pharmaceutical compositions have a fixed dose combination. In some embodiments, the pharmaceutical compositions comprise from about 0.1 mg to about 3.0 mg Colchicine; and a CXCR-2 inhibitor. In some embodiments, the pharmaceutical compositions comprise about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, or about 3.0 mg Colchicine; and a CXCR-2 inhibitor. In some embodiments, the pharmaceutical compositions comprise from about 0.1 mg to about 0.6 mg Colchicine; and a CXCR-2 inhibitor. In some embodiments, the pharmaceutical compositions comprise from about 0.1 mg to about 1.0 mg Colchicine; and a CXCR-2 inhibitor. In some embodiments, the pharmaceutical compositions comprise from about 0.5 mg to about 2.4 mg Colchicine; and a CXCR-2 inhibitor. In some embodiments, the pharmaceutical compositions comprise from about 1.2 mg to about 2.4 mg Colchicine; and a CXCR-2 inhibitor. In some embodiments, the pharmaceutical compositions comprise from about 0.6 mg to about 1.2 mg Colchicine; and a CXCR-2 inhibitor.

In some embodiments, the pharmaceutical compositions further comprise a pharmaceutically acceptable diluent or carrier.

In some embodiments, the CXCR-2 inhibitor is one of the compounds disclosed herein, or a pharmaceutically acceptable salt thereof. In some embodiments, the CXCR-2 inhibitor is N-(2-((2,3-difluorobenzyl)thio)-6-(((2R,3R)-3,4-dihydroxybutan-2-yl)amino)pyrimidin-4-yl)azetidine-1-sulfonamide (compound 1), or a pharmaceutically acceptable salt thereof. In some embodiments, the CXCR-2 inhibitor is (R)-5-((2,3-difluorobenzyl)thio)-7-((1-hydroxypropan-2-yl)amino)thiazolo[4,5-d]pyrimidin-2(3H)-one (compound 2), or a pharmaceutically acceptable salt thereof. In some embodiments, the CXCR-2 inhibitor is N-(6-(((2R,3S)-3,4-dihydroxybutan-2-yl)oxy)-2-((4-fluorobenzyl)thio)pyrimidin-4-yl)-3-methylazetidine-1-sulfonamide (compound 3) or a pharmaceutically acceptable salt thereof.

Also described herein are processes for the preparation of a pharmaceutical composition of the invention which comprise mixing a CXCR-2 inhibitor (e.g., 4), or pharmaceutically acceptable salts thereof, with a pharmaceutically acceptable adjuvant, diluent or carrier. In some embodiments, the pharmaceutical compositions are administered topically (e.g. to the lung and/or airways or to the skin) in the form of solutions, suspensions, heptafluoroalkane aerosols and dry powder formulations; or systemically, e.g. by oral administration in the form of tablets, capsules, syrups, solutions, suspensions, powders, or granules, or by parenteral administration in the form of solutions or suspensions, or by subcutaneous administration or by rectal administration in the form of suppositories or transdermally. In some embodiments, compound 3, or a pharmaceutically acceptable salt thereof, is administered orally. In some embodiments, the pharmaceutical composition is formulated for delayed release or sustained release.

### Modes of Administration

The compound, compound forms and compositions described herein are administered either alone, or in combination with, pharmaceutically acceptable adjuvants, carriers, excipients, or diluents in a pharmaceutical composition, according to standard pharmaceutical practice.

The pharmaceutical compositions described herein are, for example, in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition is, in some embodiments, in unit dosage forms suitable for single administration of precise dosages. Pharmaceutical compositions include a compound or compound form as described herein as an active ingredient, and a conventional pharmaceutical carrier or excipient. In some embodiments, these compositions include other or additional medicinal or pharmaceutical agents, carriers, adjuvants, etc.

Pharmaceutical compositions are conveniently presented in unit dosage form. In some embodiments, they are prepared with a specific amount of active compound by any of the methods well known or apparent to those skilled in the pharmaceutical arts.

### Kits

The compounds, compound forms, compositions and methods described herein provide kits for the treatment of diseases and disorders, such as the ones described herein. These kits comprise a compound, compound form, compounds, compound forms or compositions described herein in a container and, optionally, instructions teaching the use of the kit according to the various methods and approaches described herein. Such kits, in some embodiments, also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein are provided, marketed and/or promoted to health providers, including physicians, nurses, pharmacists, formulary officials, and the like. Kits are also, in some embodiments, marketed directly to the consumer.

Described herein are compositions or kits comprising a CXCR-2 inhibitor, such as any one of the compounds described herein, or the pharmaceutically acceptable salt thereof. In some embodiments, the CXCR-2 inhibitor is N-(6-(((2R,3 S)-3,4-dihydroxybutan-2-yl)oxy)-2-((4-fluorobenzyl)thio)pyrimidin-4-yl)-3-methylazetidine-1-sulfonamide (compound 3), or a pharmaceutically acceptable salt thereof. In some embodiments, the kits further comprise Colchicine.

Described herein are compositions or kits for treating a subject having been diagnosed with Familial Mediterraneen Fever (FMF) comprising a CXCR-2 inhibitor, such as any one of the compounds described herein, and instructions for administration of the CXCR-2 inhibitor to treat or prevent the Familial Mediterraneen Fever (FMF) flare-ups. In some embodiments, the CXCR-2 inhibitor is N-(6-(((2R,3 S)-3,4-dihydroxybutan-2-yl)oxy)-2-((4-fluorobenzyl)thio)pyrimidin-4-yl)-3-methylazetidine-1-sulfonamide (compound 3), or a pharmaceutically acceptable salt thereof. In some embodiments, the kits further comprise Colchicine.

Provided in certain embodiments, are compositions or kits comprising a CXCR-2 inhibitor, a double low density polyethylene plastic bag, and an HDPE container. In further embodiments, the composition or kit further comprises a foil bag (e.g., an anhydrous foil bag, such as a heat sealed anhydrous foil bag). In some embodiments, the composition or kit further comprises a desiccant; in still other embodiments, a desiccant is not necessary and/or present. In some instances, such packing improves the stability of the CXCR-2 inhibitor.

In some embodiments, the compounds, compound forms and pharmaceutical compositions described herein are utilized for diagnostics and as research reagents. For example, in some embodiments, the compounds, compound forms and pharmaceutical compositions, either alone or in combination with other compounds, are used as tools in differential and/or combinatorial analyses to elucidate expression patterns of genes expressed within cells and tissues. As one non-limiting example, expression patterns within cells or tissues treated with one or more compounds are compared to control cells or tissues not treated with compounds and the patterns produced are analyzed for differential levels of gene expression as they pertain, for example, to disease association, signaling pathway, cellular localization, expression level, size, structure or function of the genes examined. These analyses are performed on stimulated or unstimulated cells and in the presence or absence of other compounds which affect expression patterns.

### EXAMPLES

The following examples further illustrate the invention but should not be construed as in any way limiting its scope. In particular, the processing conditions are merely exemplary and can be readily varied by one of ordinary skill in the art.

All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. Unless defined otherwise, technical and scientific terms used herein have the same meaning as is commonly understood by one of skill in the art to which this invention belongs.

Embodiments of this invention are described herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited herein. Moreover, any combination of the above-described elements in all possible variations thereof is encompassed by the invention unless otherwise indicated herein or otherwise clearly contradicted by context.

### Example 1: Randomized, Double-Blind, Placebo-Controlled Phase 2 Study to Evaluate the Efficacy and Safety of Compound 3 in Subjects with Familial Mediterranean Fever Who Require a Higher Dose of Colchicine

The purpose of this study is to examine the safety and efficacy of Compound 3 in subjects who continue to have active FMF despite being on doses of 1.0 mg/day to 1.2 mg/day colchicine. This study will compare the addition of Compound 3 to increases in colchicine dose beyond 1.0 mg per day to determine which treatment results in amelioration of symptoms and fewer side effects. Impairment of neutrophil chemotaxis in combination with colchicine has the potential to improve clinical outcomes of patients with FMF not responsive to colchicine at doses of at least 1.0 mg. As there are known side effects at higher doses (>1.2 mg) of colchicine, this trial will aim to evaluate if the addition of Compound 3 in place of higher doses of colchicine ameliorates the symptoms of an active FMF flare without the side effects associated with higher doses of colchicine. In addition to either Compound 3 or a supplemental dose of colchicine, all subjects remain on their prior dose of colchicine of at least 1.0 to 1.2 mg once daily through the 24 week treatment period.

The objectives and endpoints of this study are summarized in the table below:

| Objectives | Endpoints |
|---|---|
| Primary | |
| To compare the efficacy of | Comparison of the time to the next |
| colchicine/Compound 3 | attack after resolution of the Index |
| combination therapy versus | attack |
| high dose colchicine in | |
| subjects with active FMF | |
| Secondary | |
| Evaluate the effect of | Mean number of attacks per 4 weeks |
| Compound 3 on secondary | Proportion of subjects with <1 FMF |
| measures of efficacy | attack per 4 weeks |
| compared to colchicine | Proportion of subjects with disease |
| | activity Physician Global Assessment |
| | (PGA) <2 |
| | Proportion of subjects with c-reactive |
| | protein (CRP) <10 mg/L |
| | Proportion of subjects with serum |
| | amyloid <10 mg/L |
| Evaluate the safety of | Incidence of treatment-emergent |
| Compound 3 in | adverse events (TEAEs) and serious |
| this population | TEAEs (SAEs) |
| Exploratory | |
| Evaluate the effect of | Proportion of subjects with resolution |
| Compound 3 on exploratory | of the Index attack (attack required for |
| measures of efficacy | randomization) by Day 15 |
| | Proportion of subjects who have no |
| | attacks through the Week 24 visit |
| | Change from baseline in |
| | Auto-Inflammatory Disease |
| | Activity Index (AIDAI) |
| | Patient Global Assessment of |
| | Disease Activity (PPGA) |
| | 36-Item Short Form Health Survey |
| | (SF-36) |
| | Sheehan Disability Scale (SDS) |
| Evaluate the safety of | Change from Baseline in clinical |
| Compound 3 on other measures | laboratory parameters, ECG parameters, |
| of safety and tolerability | and vital signs |
| Evaluate the pharmacokinetics | Plasma concentrations of Compound 3 |
| (PK) of Compound 3 | and its metabolites, if appropriate |
| Evaluate the pharmacodynamics | Changes from Baseline in biomarkers |
| (PD) of Compound 3 in this | and cytokines measured in blood |
| population | samples |

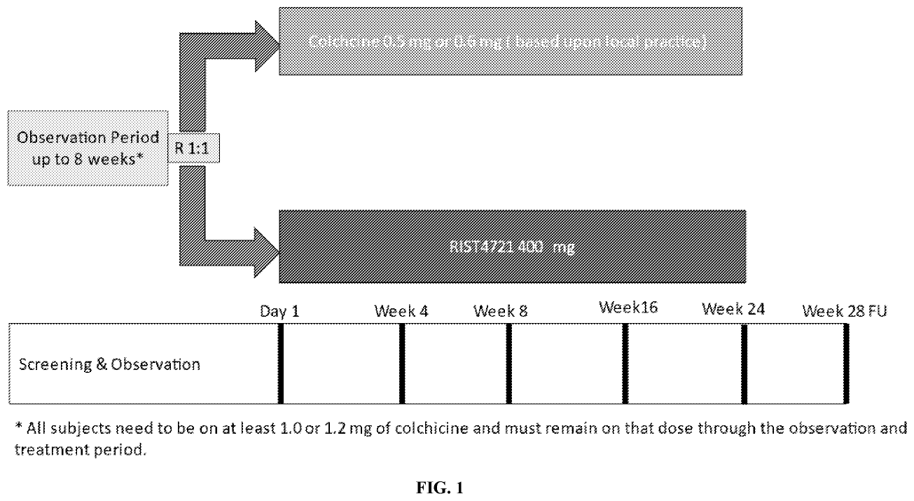

The study design is summarized in FIG. 1. This is a Phase 2, double-blind, placebo-controlled, randomized study whose purpose is to evaluate the oral administration of Compound 3 in adult subjects with active FMF (at least 1 attack per 2 months) despite being on 1.0 to 1.2 mg/day of colchicine. The study consists of a screening/observation period, a treatment period, and a follow-up period. After signing an informed consent form (JCF), subjects are observed and screened for study eligibility over at least 4 weeks and up to 8 weeks.

On Day 1 (Baseline visit), eligible subjects who also meet the randomization entry criteria are randomized 1:1 to receive the double-blind oral study treatment for 24 weeks (randomization criteria are: 1. acute FMF flare characterized by inflammation and serositis lasting approximately 12 to 72 hours prior to randomization; and 2. CRP>10 mg/L (normal CRP range≤10 mg/L)). The study treatment consists of Compound 3 400 mg (QD) and Colchicine 0.5 mg or Colchicine 0.6 mg QD. All subjects additionally remain on their prior dose of colchicine of at least 1.0 to 1.2 mg once daily through the 24 week treatment period.

After initiation of study treatment on Day 1, subjects return to the clinic and are evaluated as specified in the table below:

| | Screening/ | | | | | | | | | Notes |
|---|---|---|---|---|---|---|---|---|---|---|
| Study Week | Observation | Treatment period | | | | | Unsched- | | Follow- | End of treatment |
| Visit window | period | Baseline/ | 4 | 8 | 16 | 24 | uled | | up | (EOT); early |
| (days) | −8 to −1 | Day 1 | ±7 | ±7 | ±7 | ±7 | visit* | ET | ±7 | termination (ET) |

Screening/Administrative

| | | | | | | | | | |
|---|---|---|---|---|---|---|---|---|---|
| Informed consent | X | | | | | | | | |
| Demographics | X | | | | | | | | |
| Eligibility criteria | X | X | | | | | | | |
| FMF diagnosis | X | | | | | | | | |
| using Tel | | | | | | | | | |
| Hashomer | | | | | | | | | |
| criteria and | | | | | | | | | |
| EUROFEVER | X | | | | | | | | |
| 2020 | | | | | | | | | |
| Medical and | X | | | | | | | | Collect FMF |
| medication | | | | | | | | | attack history |
| history | | | | | | | | | for 3-6 months |
| | | | | | | | | | prior to |
| | | | | | | | | | screening |
| Height | X | | | | | | | | |
| Weight | X | X | | | | | X | X | |
| Chest X-ray | X | | | | | | | | Both |
| | | | | | | | | | posterior-anterior |
| | | | | | | | | | and lateral views, |
| | | | | | | | | | unless had a |
| | | | | | | | | | computed |
| | | | | | | | | | tomography (CT) |
| | | | | | | | | | scan within the |
| | | | | | | | | | last 4 weeks |
| Serology*/ | X | | | | | | | | *If a subject has |
| Tuberculosis t | | | | | | | | | a positive test, |
| | | | | | | | | | confirmatory |
| | | | | | | | | | serology is |
| | | | | | | | | | performed |
| Urine drug of | X | | | | | | | | |
| abuse test | | | | | | | | | |
| Pregnancy test | S | U | U | U | U | U | U | U | Serum (S); |
| | | | | | | | | | Urine (U) |

Study Treatment Administration

| | | | | | |
|---|---|---|---|---|---|
| Randomization | X | | | | |
| Study treatment | X | X | X | X | |
| dispensation | | | | | |
| Study treatment | | X | X | X | X |
| accountability | | | | | |

Safety Assessments

| | | | | | | | | | | |
|---|---|---|---|---|---|---|---|---|---|---|
| Adverse event | X | X | X | X | X | X | X | X | X | |
| Concomitant | | X | X | X | X | X | | X | X | |
| medications | | | | | | | | | | |
| Vital signs | X | X | X | X | X | X | X | X | X | |
| Supine 12-lead | X | X | X | | X | X | | X | X | Electro- |
| ECG | | | | | | | | | | cardiogram |
| | | | | | | | | | | (ECG) |

Efficacy Assessments

| | | | | | | | | | | |
|---|---|---|---|---|---|---|---|---|---|---|
| Flare/Attack | X | X | X | X | X | X | X | X | X | |
| assessments | | | | | | | | | | |
| Physician Global | X | X | X | X | X | X | | X | X | |
| Assessment of | | | | | | | | | | |
| Disease Activity | | | | | | | | | | |
| Auto- | X | X | X | X | X | X | | X | X | |
| Inflammatory | | | | | | | | | | |
| Disease Activity | | | | | | | | | | |
| Index (AIDAI) | | | | | | | | | | |
| Patient Global | X | X | X | X | X | X | | X | X | |
| Assessment of | | | | | | | | | | |
| Disease Activity | | | | | | | | | | |
| Rescue Therapy | | | | X | X | | | | | |
| Assessment | | | | | | | | | | |
| SF-36 | | X | | | | X | | X | X | 36-Item Short |
| | | | | | | | | | | Form Health |
| | | | | | | | | | | Survey (SF-36) |
| Sheehan Disability | X | X | | X | | X | | X | X | |
| Scale (SDS) | | | | | | | | | | |

Clinical Laboratory Assessments

| | | | | | | | | | | |
|---|---|---|---|---|---|---|---|---|---|---|
| Safety | X | X | X | X | X | X | | X | X | Includes |
| laboratories | | | | | | | | | | hematology, |
| | | | | | | | | | | serum chemistry |
| | | | | | | | | | | and urinalysis. |
| C-reactive protein | X | X | X | X | X | X | X | X | X | |
| Serum amyloid A | X | X | X | X | X | X | X | X | X | |
| Biomarkers | | X | X | X | X | X | | X | X | |

Pharmacokinetics

| | | | | | | | | |
|---|---|---|---|---|---|---|---|---|
| Serum sample | X | X | X | X | X | X | X | Blood samples |
| for Compound 3 | | | | | | | | for sparse |
| concentration | | | | | | | | pharmacokinetics |
| | | | | | | | | are collected |
| | | | | | | | | predose |

*Additional assessment including safety lab as clinically indicated

Subjects who permanently discontinue study treatment and continue in the study complete the early termination (ET) visit at the time of study treatment discontinuation and then complete remaining study visits. At subsequent visits, all study procedures are completed except for dispensation/return and accountability of study treatment. Subjects who withdraw from the study, regardless of the reason, are requested to return to the clinic to complete the ET visit. All subjects who remain on study treatment through and including Week 24 are asked to return for a Follow-up visit approximately 4 weeks after their last dose of study treatment to assess safety.

Study treatment is administered according to the table below:

| Investigational Product | | |
|---|---|---|
| Name | Active: Compound 3 | Control: Colchicine |
| Dose Formulation | | Matching tablets containing colchicine |
| | | instead of Compound 3 |
| Unit Dose Strength(s) | 400 mg per tablet of Compound 3 | Colchicine 0.5 mg or Colchicine 0.6 |
| | 200 mg per tablet of Compound 3 | mg |
| Dose regimen | 1 tablet QD | 1 tablet QD |

| | |
|---|---|
| Route of Administration | Oral. |
| and Instructions | On Day 1, the clinical study team instructs the subject how to administer |
| | the study treatment |
| Sourcing | Study treatment is provided to the site centrally by the Sponsor or |
| | designated representative. |
| Packaging | Compound 3 and Colchicine tablets are supplied in bottles. |
| Labelling | Label text at a minimum includes the protocol number, lot number, storage |
| | conditions, and Sponsor name and address. Labels comply with local |
| | regulatory requirements for study treatments. Label recites that Compound |
| | 3 is indicated for treatment of FMF flares in patents who do not experience |
| | amelioration of symptoms with a dose of 1.0 or 1.2 mg colchicine, or who |
| | experience significant side effects with a greater than 1.2 mg dose of |
| | colchicine. |

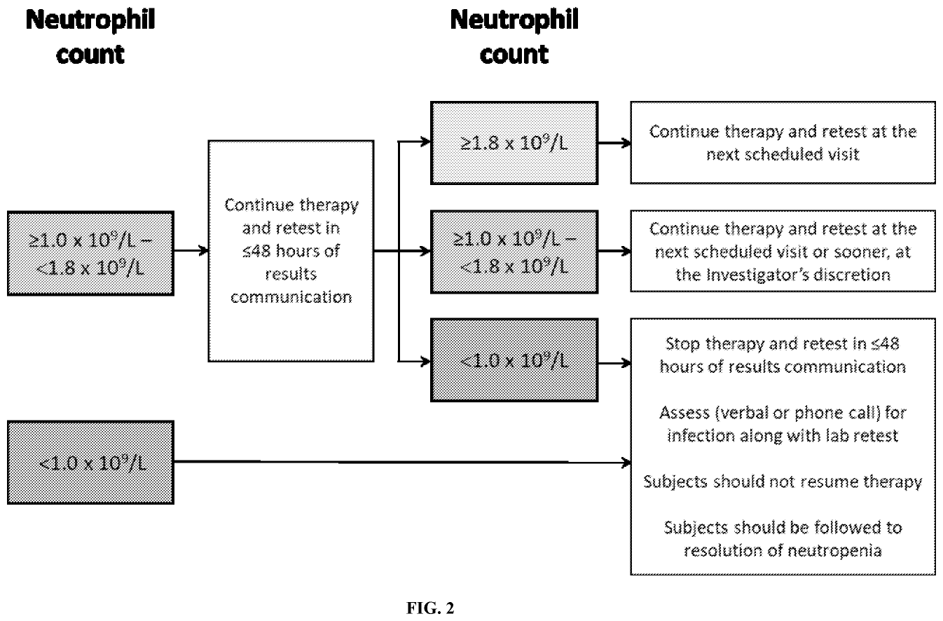

Absolute and relative neutrophil and WBC count results are only disclosed to the respective investigators if ANC<1.8×109/L, in which case immediate actions are taken. Retest results are communicated to the investigators who follow up on neutropenia and the incidence of infections with subjects having absolute neutrophil count results<1.0×109/L. Neutropenia is followed up through resolution. The steps for addressing neutrophil counts are summarized in FIG. 2.

Dose Interruption of study treatment is undertaken for confirmed ANC results<1.0×10⁹/L. Once the ANC result is above 1.8×10⁹/L, subjects re-initiate blinded study treatment by reducing the study treatment dose as follows: for subjects who are on colchicine (1 tablets), they will receive colchicine (1 tablets); for subjects who are on Compound 3 400 mg (1 tablets), they will receive Compound 3 200 mg dose (1 tablets of Compound 3 200 mg). Subjects are requested to attend an unscheduled visit after dose reduction to collect blood samples for safety and plasma concentrations, if possible. For subjects who have already performed dose reduction: If a subject experiences another case of ANC results<1.0×10⁹/L, they interrupt dosing for the remainder of the study.

The clinical laboratory tests performed are summarized in the table below:

| Laboratory Testing | Tests Included |
|---|---|
| Hematology | aPTT, HCT, Hgb, INR, MCH, MCHC, MCV, MPV, PLT, PT, RBC, WBC, |
| | and differentials (neutrophils, lymphocytes, monocytes, eosinophils, and |
| | basophils relative and absolute) |
| Biochemistry | Albumin, alkaline phosphatase, ALT, AST, chloride, cholesterol |
| | (nonfasting), creatinine (enzymatic), GGT, glucose random, hs-CRP, LDH, |
| | potassium, sodium, total bilirubin, triglycerides, blood urea nitrogen (BUN), |
| | uric acid |
| Urinalysis | Dipstick and microscopic analysis |
| Urine pregnancy test | For WOCBP (at each visit, except screening) |
| Laboratory tests | FSH levels for females who have had a cessation of menses for at |
| required at screening | least 12 months without an alternative medical cause |
| only | β-hCG for WOCBP (screening only) |
| | Tuberculosis test (PPD or QuantiFERON-TB Gold) |
| | Serology (HBV [HBsAg, anti-HBc], HCV, HIV) |
| | Drugs of abuse (amphetamines, methamphetamines, cocaine, |
| | and phencyclidine) |
| Additional Laboratory | C-reactive protein (CRP) by local laboratory |
| tests | Serum amyloid A (SAA) by central laboratory |

Abbreviations: ALT, alanine aminotransferase; anti-HBc, antibody to hepatitis B core antigen; aPTT, activated partial thromboplastinltime; AST, aspartate aminotransferase; β-hCG, β-human chorionic gonadotropin; BUN, blood urea nitrogen; FSH, follicle-stimulating hormone; GGT, gamma-glutamyl-transferase; HBsAg, hepatitis B surface antigens; HBV, hepatitis B virus; HCT, hematocrit; HCV, hepatitis C virus; Hgb, hemoglobin; HJV, human immunodeficiency vimus; hs-CRP, high-sensitivity C-reactive protein; JNR, international normalized ratio; LDH, lactate dehydrogenase; MCH, mean corpuscular hemoglobin; MCHC, mean corpuscular hemoglobin concentration; MCV, mean corpuscular volume; MVPV, mean platelet volume; PLT, platelets; PPD, purified protein derivative; PT, prothrombin time; RBC, red blood cell (count); WBC, white blood cell (count); WOCBP, women of childbearing potential.

Blood samples are collected for measurement of plasma concentrations of Compound 3 and its metabolites, if applicable, as specified in the table below:

| | | | PK | PK Collection | |
|---|---|---|---|---|---|
| Timepoint | Dose | Time | Collection | Time Window | |
| Day 1/Baseline | morning | predose | X | −1 | hour |
| | | 2 hours | X | ±10 | minutes |
| | | postdose | | | |
| Week 24 | morning | predose | X | −1 | hour |
| | | 2 hours | X | ±10 | minutes |
| | | postdose | | | |
| ET/unscheduled | none | anytime | X | | |
| visit any time | | | | | |
| after Baseline | | | | | |

Abbreviations: ET, early termination visit

PK samples are analyzed with a validated method; for metabolites, the samples are analyzed with a fit-for-purpose method(s). The samples are used for metabolite profiling or bioanalytical method development and validation. The samples are also used for measuring concentrations of colchicine.

What is claimed is:

1. A method for treating Familial Mediterranean Fever (FMF) in a subject in need thereof, comprising administering to the subject a combination of:
(i) Colchicine, or a pharmaceutically acceptable salt thereof, and
(ii) a CXCR-2 inhibitor, or a pharmaceutically acceptable salt thereof.

2. A method for preventing Familial Mediterranean Fever (FMF) flare-ups in a subject having been diagnosed with FMF, comprising administering to the subject a combination of:
(i) Colchicine, or a pharmaceutically acceptable salt thereof, and
(ii) a CXCR-2 inhibitor, or a pharmaceutically acceptable salt thereof.

3. A method for treating acute Familial Mediterranean Fever (FMF) flare-ups in a subject having been diagnosed with FMF, comprising administering to the subject a combination of:
(i) Colchicine, or a pharmaceutically acceptable salt thereof; and
(ii) a CXCR-2 inhibitor, or a pharmaceutically acceptable salt thereof.

4. A method for decreasing the severity of Familial Mediterranean Fever (FMF) flare-ups in a subject having been diagnosed with FMF, comprising administering to the subject a combination of:
(i) Colchicine, or a pharmaceutically acceptable salt thereof, and
(ii) a CXCR-2 inhibitor, or a pharmaceutically acceptable salt thereof.

5. The method of claim 4, wherein the severity of the FMF flare-ups is decreased as compared to a subject being administered Colchicine alone.

6. The method of claim 4, wherein the severity of the FMF flare-ups is decreased as compared to a subject receiving no therapy.

7. The method of claim 5 or 6, wherein the severity of the flare-ups is decreased by at least about 50%.

8. The method of claim 5 or 6, wherein the severity of the flare-ups is decreased by at least about 70%.

9. The method of claim 5 or 6, wherein the severity of the flare-ups is decreased by at least about 90%.

10. The method of claim 5 or 6, wherein the severity of the flare-ups is decreased by at least about 99%.

11. A method for decreasing the occurrence of Familial Mediterranean Fever (FMF) flare-ups in a subject having been diagnosed with FMF, comprising administering to the subject a combination of:
(i) Colchicine, or a pharmaceutically acceptable salt thereof, and
(ii) a CXCR-2 inhibitor, or a pharmaceutically acceptable salt thereof.

12. The method of claim 11, wherein the occurrence of the FMF flare-ups is decreased as compared to a subject being administered Colchicine alone.

13. The method of claim 11, wherein the occurrence of the FMF flare-ups is decreased as compared to a subject receiving no therapy.

14. The method of claim 12 or 13, wherein the occurrence of the flare-ups is decreased by at least about 50%.

15. The method of claim 12 or 13, wherein the occurrence of the flare-ups is decreased by at least about 70%.

16. The method of claim 12 or 13, wherein the occurrence of the flare-ups is decreased by at least about 90%.

17. The method of claim 12 or 13, wherein the occurrence of the flare-ups is decreased by at least about 99%.

18. The method of any one of claim 1-17, wherein the combination is a synergistic combination.

19. The method of any one of claim 1-18, wherein the CXCR-2 inhibitor is N-(6-(((2R,3S)-3,4-dihydroxy butan-2-yl)oxy)-2-((4-fluoro benzyl)thio)pyrimidin-4-yl)-3-methylazetidine-1-sulfonamide: 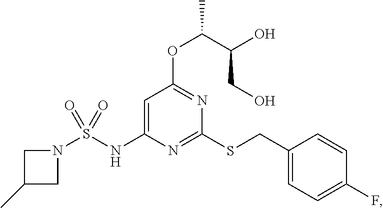 [SMILES: CC1CN(S(=O)(=O)Nc2cc(O[C@H](C)[C@@H](O)CO)nc(SCc3ccc(F)cc3)n2)C1]
or a pharmaceutically acceptable salt thereof.

20. The method of any one of claims 1-19, wherein the subject is colchicine-resistant.

21. The method of claim 20, wherein colchicine-resistant means the subject has stopped responding to Colchicine treatment.

22. The method of claim 20, wherein colchicine-resistant means more than 1 flare-up every 3 months despite treatment with 2 mg/day of colchicine.

23. The method of claim 20, wherein colchicine-resistant means one flare-up or more per month in a subject who is receiving the maximally Colchicine tolerated dose for six months or more.

24. The method of claim 20, wherein colchicine-resistant means more than 6 flare-ups per year.

25. The method of claim 20, wherein colchicine-resistant means more than 3 flare-ups over 4-6 months.

26. The method of any one of claims 2-25, wherein the FMF flare-ups comprise fever, abdominal pain, joint pain, pleuritis, pericarditis, or any combinations thereof.

27. The method of any one of claims 2-26, wherein the FMF flare-ups comprise fever.

28. The method of any one of claims 2-26, wherein the FMF flare-ups comprise abdominal pain.

29. The method of any one of claims 2-26, wherein the FMF flare-ups comprise joint pain.

30. The method of any one of claims 1-29, wherein colchicine is administered in a therapeutically effective amount.

31. The method of any one of claims 1-29, wherein colchicine is administered in a sub-therapeutically effective amount.

32. The method of claim 31, wherein the sub-therapeutic amount of colchicine results in fewer side effects than a therapeutic amount of colchicine.

33. The method of claim 30 or 31, wherein the amount of Colchicine, or a pharmaceutically acceptable salt thereof, is between about 0.3 mg/day and about 3.0 mg/day.

34. The method of claim 30 or 31, wherein the amount of Colchicine, or a pharmaceutically acceptable salt thereof, is between about 0.6 mg/day and about 2.4 mg/day.

35. The method of claim 30 or 31, wherein the amount of Colchicine, or a pharmaceutically acceptable salt thereof, is between about 1.2 mg/day and about 2.4 mg/day.

36. The method of claim 30 or 31, wherein the amount of Colchicine, or a pharmaceutically acceptable salt thereof, is about 0.6 mg/day.

37. The method of claim 30 or 31, wherein the amount of Colchicine, or a pharmaceutically acceptable salt thereof, is about 1.2 mg/day.

38. The method of claim 30 or 31, wherein the amount of Colchicine, or a pharmaceutically acceptable salt thereof, is about 2.4 mg/day.

39. The method of claim 30 or 31, wherein the amount of Colchicine, or a pharmaceutically acceptable salt thereof, is about 0.5 mg/day.

40. The method of claim 30 or 31, wherein the amount of Colchicine, or a pharmaceutically acceptable salt thereof, is about 1.0 mg/day.

41. The method of claim 30 or 31, wherein the amount of Colchicine, or a pharmaceutically acceptable salt thereof, is about 1.5 mg/day.

42. The method of claim 30 or 31, wherein the amount of Colchicine, or a pharmaceutically acceptable salt thereof, is about 2.0 mg/day.

43. The method of claim 30 or 31, wherein the amount of Colchicine, or a pharmaceutically acceptable salt thereof, is about 2.5 mg/day.

44. The method of claim 30 or 31, wherein the amount of Colchicine, or a pharmaceutically acceptable salt thereof, is about 3.0 mg/day.

45. The method of any one of claims 1-44, wherein the CXCR-2 inhibitor is administered in a therapeutically effective amount.

46. The method of any one of claims 1-44, wherein the CXCR-2 inhibitor is administered in a sub-therapeutically effective amount.

47. The method of claim 45 or 46, wherein the amount of the CXCR-2 inhibitor, or a pharmaceutically acceptable salt thereof, is between about 10 mg/day and about 500 mg/day.

48. The method of claim 47, wherein the amount of the CXCR-2 inhibitor, or a pharmaceutically acceptable salt thereof, is about 200 mg/day.

49. The method of claim 47, wherein the amount of the CXCR-2 inhibitor, or a pharmaceutically acceptable salt thereof, is about 300 mg/day.

50. The method of claim 47, wherein the amount of the CXCR-2 inhibitor, or a pharmaceutically acceptable salt thereof, is about 400 mg/day.

51. The method of any one of claims 1-50, wherein the CXCR-2 inhibitor, or a pharmaceutically acceptable salt thereof, and Colchicine, or a pharmaceutically acceptable salt thereof are formulated in the same pharmaceutical composition.

52. The method of claim 51, wherein the pharmaceutical composition is a solution, suspension, tablet, or capsule.

53. The method of claim 52, wherein the pharmaceutical composition is formulated as a sustained release or delayed release formulation.

54. The method of any one of claims 1-50, wherein the CXCR-2 inhibitor, or a pharmaceutically acceptable salt thereof, and Colchicine, or a pharmaceutically acceptable salt thereof are formulated in different pharmaceutical compositions.

55. The method of claim 54, wherein the Colchicine pharmaceutical composition is a solution, suspension, tablet, or capsule.

56. The method of claim 54, wherein the Colchicine pharmaceutical composition is formulated as a sustained release or delayed release formulation.

57. The method of claim 54, wherein the CXCR-2 inhibitor pharmaceutical composition is a solution, suspension, tablet, or capsule.

58. The method of claim 54, wherein the CXCR-2 inhibitor pharmaceutical composition is formulated as a sustained release or delayed release formulation.

59. The method of any one of claims 1-50, wherein the CXCR-2 inhibitor and Colchicine are administered concomitantly.

60. The method of any one of claims 1-50, wherein the CXCR-2 inhibitor and Colchicine are administered sequentially.

61. The method of any one of claims 2-60, wherein the FMF flare-up is an acute FMF flare-up.

62. The method of any one of claims 2-61, wherein the subject presents with a C-reactive protein (CRP) level of greater than 10 mg/L.
